(19) United States
(12) Patent Application Publication
(10) Pub. No.: US 2022/0007861 A1
(43) Pub. Date: Jan. 13, 2022

(54) PLAGIOCEPHALY PILLOW ASSEMBLY, PLAGIOCEPHALY PREVENTION SET AND METHOD FOR PREVENTING DEFORMATIONAL PLAGIOCEPHALY

(71) Applicant: GROUPE BBLUV INC., Montreal (CA)

(72) Inventor: Mathieu Tremblay, Montreal (CA)

(21) Appl. No.: 17/296,296

(22) PCT Filed: Nov. 19, 2019
(86) PCT No.: PCT/CA2019/051649

§ 371 Date: May 24, 2021

Related U.S. Application Data
(60) Provisional application No. 62/770,925, filed on Nov. 23, 2018.

(51) Int. Cl. A47G 9/10; A61F 5/058; A47D 15/00

(52) U.S. Cl. CPC A47G 9/10; A47D 15/00; A61F 5/05891

(57) ABSTRACT

The present disclosure concerns a plagiocephaly pillow assembly for preventing deformational plagiocephaly of a head of an infant. The plagiocephaly pillow assembly comprises a first resilient layer with a first set of depressions formed therein and a second resilient layer superposable onto the first resilient layer and comprising a top surface configured to support a portion of the head of the infant, a second set of depressions being formed in the second resilient layer. At least some of the depressions of one of the first and second sets of depressions are at least partially offset with regards to the depressions of the other of the first and second sets of depressions when the first and second resilient layers are superposed onto each other. The present disclosure also concerns a plagiocephaly prevention set and a method for preventing deformational plagiocephaly of a head of an infant.

### PRIOR APPLICATION

The present application claims priority from U.S. provisional patent application No. 62/770,925, filed on Nov. 23, 2018, and entitled “PLAGIOCEPHALY PILLOW ASSEMBLY”, the disclosure of which being hereby incorporated by reference in its entirety.

### TECHNICAL FIELD

The technical field relates to a device for preventing deformational plagiocephaly. More particularly, the invention relates to a plagiocephaly pillow assembly for preventing deformational plagiocephaly of a head of an infant, to a plagiocephaly prevention set and to a method for preventing deformational plagiocephaly of a head of an infant.

### BACKGROUND

Since a few decades, it has become more common for an infant to be laid on their back (i.e. face-up) for a substantial amount of time. For example, an infant is placed on their back to sleep, as well as to entertain themselves.

Because young infants—especially when they are premature—have relatively soft and malleable head's bones, the weight of the infant's head, when they are laid on their back for a substantial amount of time, can gradually cause a deformation in the back of the skull, called deformational plagiocephaly, or on at least one of the sides of the skull, called deformational brachycephaly.

Some devices exist, that are configured to be placed under the back of the head of the infant, so as to limit the risk of deformational plagiocephaly and/or brachycephaly. However, such devices might not be adapted to the morphology of the head of the infant and/or might not be stably kept in place when the infant is laid on their back for a substantial amount of time.

In view of the above, there is a need for a device which would be able to overcome or at least minimize some of the above-discussed prior art concerns.

### BRIEF SUMMARY

It is therefore an aim of the present invention to address the above-mentioned issues.

According to a general aspect, there is provided a plagiocephaly pillow assembly for preventing deformational plagiocephaly of a head of an infant, the plagiocephaly pillow assembly comprising: a first resilient layer with a first set of depressions formed therein; a second resilient layer superposable onto the first resilient layer and comprising a top surface configured to support a portion of the head of the infant, a second set of depressions being formed in the second resilient layer. At least some of the depressions of one of the first and second sets of depressions are at least partially offset with regards to the depressions of the other of the first and second sets of depressions when the first and second resilient layers are superposed onto each other.

According to another general aspect, there is provided a plagiocephaly pillow assembly for preventing deformational plagiocephaly of a head of an infant, the plagiocephaly pillow assembly comprising: a first resilient layer having: a top surface; and a bottom surface, opposed to the top surface; a first set of depressions being formed in the first resilient layer, extending at least partially between the top and bottom surfaces and defining a first depression pattern having a width, a length and a surface area; a second resilient layer superposable onto the first resilient layer having: a top surface configured to support a portion of the head of the infant; and a bottom surface, opposed to the top surface and facing the top surface of the first resilient layer when the first and second layers are superposed onto each other; a second set of depressions being formed in the second resilient layer, extending at least partially between the top and bottom surfaces and defining a second depression pattern having a width, a length and a surface area. The second depression pattern is different from the first depression pattern by at least one of the width, the length, the surface area and the number, the dimensions and the locations of the depressions.

According to another general aspect, there is provided a plagiocephaly pillow assembly for preventing deformational plagiocephaly of a head of an infant, the plagiocephaly pillow assembly comprising: a first resilient layer with a first set of depressions formed therein; a second resilient layer superposable onto the first resilient layer and comprising a top surface configured to support a portion of the head of the infant, a second set of depressions being formed in the second resilient layer. A density of the first resilient layer is different from a density of the second resilient layer.

According to another general aspect, there is provided a plagiocephaly prevention set comprising: a plagiocephaly pillow assembly according to the present disclosure; and at least one of a cushion superposable onto the top surface of the second resilient layer and a wrapping cover shaped and dimensioned to at least partially wrap the infant once the head of the infant is supported on the top surface of the second resilient layer.

According to another general aspect, there is provided a method for preventing deformational plagiocephaly of a head of an infant comprising: providing a plagiocephaly prevention set comprising: a first resilient layer with a first set of depressions formed therein; a second resilient layer comprising a top surface, a second set of depressions being formed in the second resilient layer; at least some of the depressions of one of the first and second sets of depressions being at least partially offset with regards to the depressions of the other of the first and second sets of depressions when the first and second resilient layers are superposed onto each other; superposing the second resilient layer onto the first resilient layer; positioning the plagiocephaly pillow assembly on a surface; and placing the infant face-up on the top surface of the second resilient layer, at least a portion of a back of the head of the infant being supported on a section of the top surface of the second resilient layer comprising said second set of depressions.

According to another general aspect, there is provided a plagiocephaly pillow assembly for preventing deformational plagiocephaly of a head of an infant. The plagiocephaly pillow assembly comprises a first resilient layer having a first set of depressions formed therein, a second resilient layer superposable onto the resilient first layer and configured to support a portion of the head of the infant. The second resilient layer has a second set of depressions formed therein. At least some of the depressions of one of the first and second sets are at least partially offset with regards to the depressions of the other of the first and second sets when the first and second resilient layers are superposed.

According to another general aspect, there is provided a plagiocephaly pillow assembly for preventing deformational plagiocephaly of a head of an infant. The plagiocephaly pillow assembly comprises a first resilient layer having a top surface, and a bottom surface, opposed to the top surface. A first set of depressions extend at least partially between the top and bottom surfaces and defining a first depression pattern. The plagiocephaly pillow assembly further comprises a second resilient layer superposable onto the first resilient layer having a top surface configured to support a portion of the head of the infant, and a bottom surface, opposed to the top surface and facing the top surface of the first resilient layer when the first and second layers are superposed. A second set of depressions extend at least partially between the top and bottom surfaces and define a second depression pattern. The second depression pattern is different from the first depression pattern by at least one of the number, the dimensions and the locations of the depressions.

According to another general aspect, there is provided a plagiocephaly pillow assembly for preventing deformational plagiocephaly of a head of an infant. The plagiocephaly pillow assembly comprises a first resilient layer having a first set of depressions formed therein. The first set of depressions has a first surface area. The plagiocephaly pillow assembly further comprises a second resilient layer superposable onto the resilient first layer and configured to support a portion of the head of the infant. The second resilient layer has a second set of depressions formed therein. The second set of depressions has a second surface area. The second surface area is greater than the first surface area.

According to another general aspect, there is provided a method for preventing deformational plagiocephaly of a head of an infant comprising providing a plagiocephaly pillow assembly according to the present disclosure, positioning the plagiocephaly pillow assembly on a surface and placing the infant face-up on the second resilient layer. At least a portion of a back of the head of the infant is supported on a section of the second resilient layer comprising the second set of depressions.

### BRIEF DESCRIPTION OF THE DRAWINGS

FIG. 1 is a top perspective view of a plagiocephaly prevention set in accordance with an embodiment;

FIG. 2 is a top perspective view of a plagiocephaly pillow assembly of the plagiocephaly prevention set of FIG. 1, the plagiocephaly pillow assembly comprising a pillowcase configured in an open configuration, a base resilient layer and first and second resilient layers being arranged in the pillowcase;

FIG. 3 is a top perspective view of the plagiocephaly pillow assembly of FIG. 2, the pillowcase being configured in a closed configuration;

FIG. 4 is a top plan view of the second resilient layer of the plagiocephaly pillow assembly of FIG. 2;

FIG. 5 is a top plan view of the first resilient layer of the plagiocephaly pillow assembly of FIG. 2;

FIG. 6 is a top plan view of the base resilient layer of the plagiocephaly pillow assembly of FIG. 2;

FIG. 7 is a top plan view of a cushion of the plagiocephaly prevention set of FIG. 1;

FIG. 8 is a bottom plan view of the pillowcase of FIG. 2;

FIG. 9 is a top plan view of a wrapping cover of the plagiocephaly prevention set of FIG. 1;

FIG. 10A is a top plan view of a superposition of the second and first resilient layers of the plagiocephaly pillow assembly of FIG. 2;

FIG. 10B is a top plan view of a superposition of the first and base resilient layers of the plagiocephaly pillow assembly of FIG. 2;

FIG. 10C is a bottom plan view of a superposition of the second and base resilient layers of the plagiocephaly pillow assembly of FIG. 2;

FIG. 11 is a diagram summarizing different steps of a method for preventing deformational plagiocephaly of a head of an infant;

FIG. 12 is a top plan view of a second resilient layer of a plagiocephaly pillow assembly in accordance with another embodiment;

FIG. 13 is a top perspective view of a plagiocephaly prevention set in accordance with another embodiment;

FIG. 14 is a top perspective view of a plagiocephaly pillow assembly of the plagiocephaly prevention set of FIG. 13, the plagiocephaly pillow assembly comprising a pillowcase configured in the open configuration, a base resilient layer and first and second resilient layers being arranged in the pillowcase;

FIG. 15 is a top perspective view of the plagiocephaly pillow assembly of FIG. 14, the pillowcase being configured in the closed configuration;

FIG. 16 is a top plan view of the second resilient layer of the plagiocephaly pillow assembly of FIG. 14;

FIG. 17 is a top plan view of the first resilient layer of the plagiocephaly pillow assembly of FIG. 14;

FIG. 18 is a top plan view of the base resilient layer of the plagiocephaly pillow assembly of FIG. 14;

FIG. 19 is a bottom plan view of a cushion of the plagiocephaly prevention set of FIG. 13;

FIG. 20 is a bottom plan view of the pillowcase of FIG. 14;

FIG. 21 is a top plan view of a wrapping cover of the plagiocephaly prevention set of FIG. 13;

FIG. 22A is a top plan view of a superposition of the second and first resilient layers of the plagiocephaly pillow assembly of FIG. 14;

FIG. 22B is a top plan view of a superposition of the first and base resilient layers of the plagiocephaly pillow assembly of FIG. 14;

FIG. 22C is a bottom plan view of a superposition of the second and base resilient layers of the plagiocephaly pillow assembly of FIG. 14; and

FIGS. 23A to 23E are representations of the different steps of the method for preventing deformational plagiocephaly of a head of an infant using the plagiocephaly prevention set of FIG. 13.

### DETAILED DESCRIPTION

In the following description, the same numerical references refer to similar elements. Furthermore, for the sake of simplicity and clarity, namely so as to not unduly burden the figures with several references numbers, not all figures contain references to all the components and features, and references to some components and features may be found in only one figure, and components and features of the present disclosure which are illustrated in other figures can be easily inferred therefrom. The embodiments, geometrical configurations, materials mentioned and/or dimensions shown in the figures are optional and are given for exemplification purposes only.

Moreover, it will be appreciated that positional descriptions such as “above”, “below”, “forward”, “rearward” “left”, “right” and the like should, unless otherwise indicated, be taken in the context of the figures only and should not be considered limiting. Moreover, the figures are meant to be illustrative of certain characteristics of the plagiocephaly prevention set and are not necessarily to scale.

To provide a more concise description, some of the quantitative expressions given herein may be qualified with the term “about”. It is understood that whether the term “about” is used explicitly or not, every quantity given herein is meant to refer to an actual given value, and it is also meant to refer to the approximation to such given value that would reasonably be inferred based on the ordinary skill in the art, including approximations due to the experimental and/or measurement conditions for such given value.

In the following description, an embodiment is an example or implementation. The various appearances of “one embodiment,” “an embodiment” or “some embodiments” do not necessarily all refer to the same embodiments. Although various features may be described in the context of a single embodiment, the features may also be provided separately or in any suitable combination. Conversely, although the invention may be described herein in the context of separate embodiments for clarity, it may also be implemented in a single embodiment. Reference in the specification to “some embodiments”, “an embodiment”, “one embodiment” or “other embodiments” means that a particular feature, structure, or characteristic described in connection with the embodiments is included in at least some embodiments, but not necessarily all embodiments.

It is to be understood that the phraseology and terminology employed herein is not to be construed as limiting and are for descriptive purpose only. The principles and uses of the teachings of the present disclosure may be better understood with reference to the accompanying description, figures and examples. It is to be understood that the details set forth herein do not construe a limitation to an application of the disclosure.

Furthermore, it is to be understood that the disclosure can be carried out or practiced in various ways and that the disclosure can be implemented in embodiments other than the ones outlined in the description above. It is to be understood that the terms “including”, “comprising”, and grammatical variants thereof do not preclude the addition of one or more components, features, steps, or integers or groups thereof and that the terms are to be construed as specifying components, features, steps or integers. If the specification or claims refer to “an additional” element, that does not preclude there being more than one of the additional element. It is to be understood that where the claims or specification refer to “a” or “an” element, such reference is not be construed that there is only one of that element. It is to be understood that where the specification states that a component, feature, structure, or characteristic “may”, “might”, “can” or “could” be included, that particular component, feature, structure, or characteristic is not required to be included.

The descriptions, examples, methods and materials presented in the claims and the specification are not to be construed as limiting but rather as illustrative only. Meanings of technical and scientific terms used herein are to be commonly understood as by one of ordinary skill in the art to which the invention belongs, unless otherwise defined. It will be appreciated that the methods described herein may be performed in the described order, or in any suitable order.

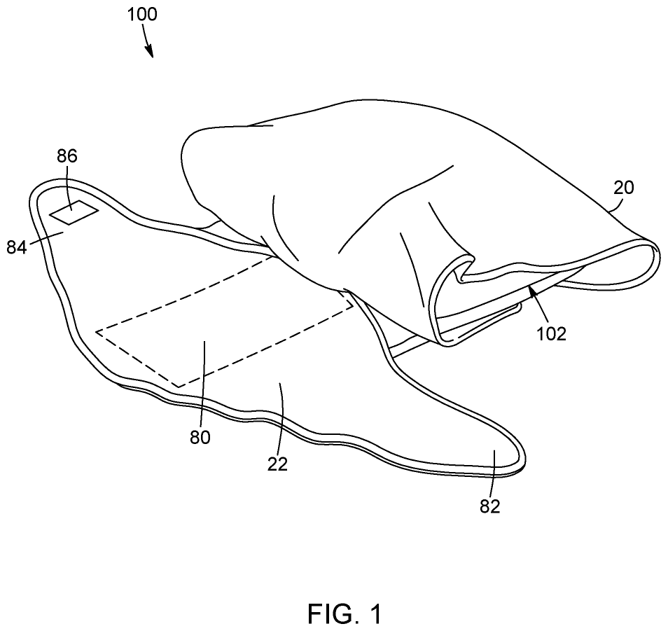

Referring now to the drawings, and more particularly to FIGS. 1 to 10C, there is shown a plagiocephaly prevention set 100 that is configured to prevent or at least limit deformational plagiocephaly of a head of an infant.

In the embodiment shown, the plagiocephaly prevention set 100 is configured to be used with an infant being less than about 1 year old, a weight of the head of the infant being comprised between about 0.2 kg and about 1.5 kgs. The plagiocephaly prevention set 100 might for instance be used with newborn children being only a few weeks old or a few months old, and/or with new born premature babies.

In the embodiment shown, the plagiocephaly prevention set 100 comprises a plagiocephaly pillow assembly 102 comprising a superposition of a base resilient layer 10 and first and second resilient layers 12, 14. The different resilient layers 10, 12, 14 are arranged together in a superposed configuration in a pillowcase 16. It is understood that in the embodiment shown in which the plagiocephaly pillow assembly 102 comprises three superposed resilient layers 10, 12, 14, the first resilient layer 12 forms an intermediate or median layer, whereas the second resilient layer 14 forms a top layer. In the following description, the term “superposed” should be understood as meaning that the different resilient layers 10, 12, 14 are arranged substantially vertically over each other. Moreover, the term “superposed” should be understood as meaning that different elements are arranged substantially over each other, either directly or indirectly (i.e. an intermediate component could be arranged between two superposed elements).

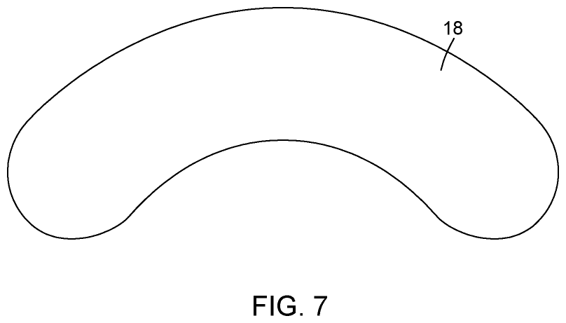

As best seen in FIG. 7, the plagiocephaly prevention set 100 can further comprise a head-surrounding cushion 18 (or cushion 18) configurable on the plagiocephaly pillow assembly 102. The plagiocephaly prevention set 100 can also comprise a protection cover 20 (FIG. 1) surrounding (or at least partially surrounding) the cushion 18 and the plagiocephaly pillow assembly 102, and a wrapping cover 22 to be wrapped at least partially around the infant when the infant is placed face-up on the plagiocephaly pillow assembly 102.

### Plagiocephaly Pillow Assembly

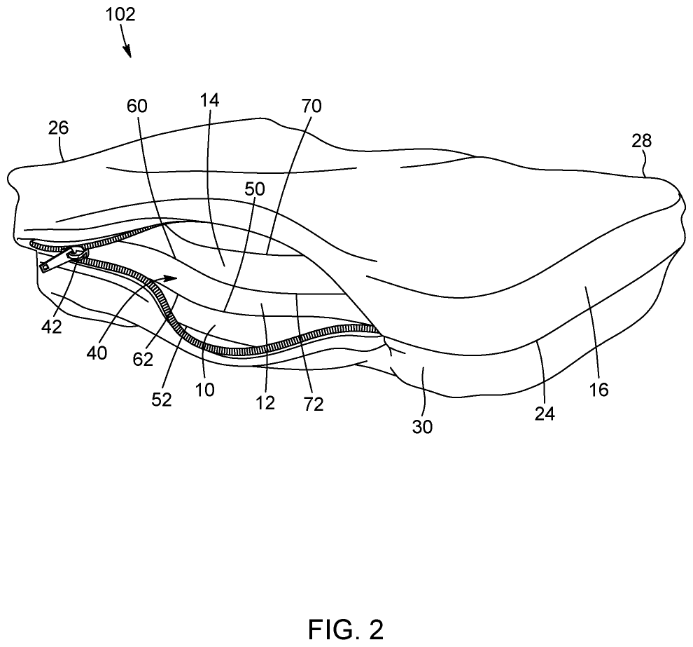

In the embodiment shown, and as represented in particular in FIG. 2, the plagiocephaly pillow assembly 102 comprises three superposed resilient layers 10, 12, 14, but a plagiocephaly pillow assembly comprising only two superposed resilient layers, or more than three superposed resilient layers could also be conceived.

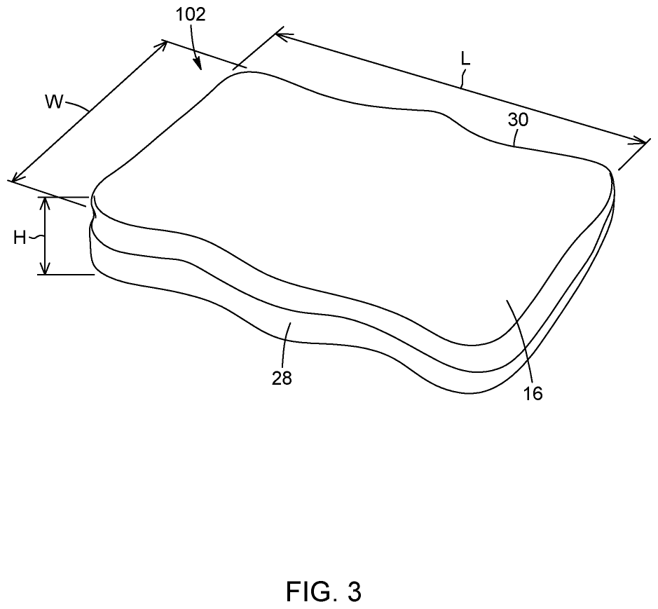

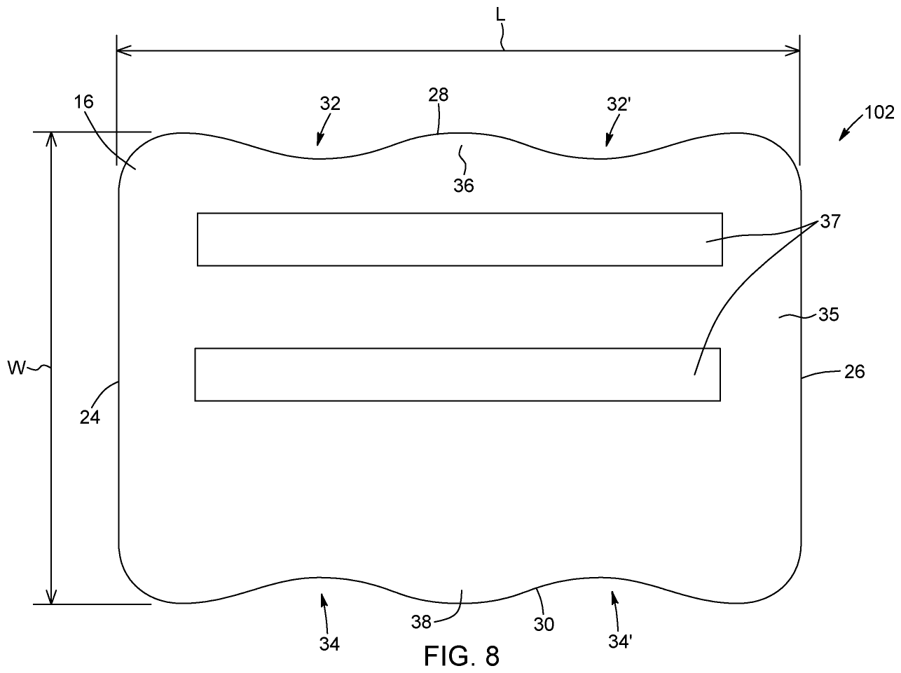

In the embodiment shown, as represented in FIGS. 3 and 8, the plagiocephaly pillow assembly 102 has a substantially parallelepiped shape having a length L, a width W, both being considered in a substantially horizontal plane, and a height H, considered along a substantially vertical direction. The plagiocephaly pillow assembly 102 is dimensioned to receive and support at least a portion of the head of the infant. The terms “length” and “width” should be understood with respect to a longitudinal direction defined by a body of the infant having their head at least partially supported by the plagiocephaly pillow assembly 102, i.e. length is intended to mean substantially normal to the longitudinal direction defined by the infant body, while width is intended to mean substantially parallel to the longitudinal direction defined by the infant body.

In some embodiments, the length L of the plagiocephaly pillow assembly 102 is comprised between about 100 mm and about 700 mm. In some other embodiments, the length L of the plagiocephaly pillow assembly 102 is comprised between about 200 mm and about 500 mm. In some other embodiments, the length L is comprised between about 250 mm and about 400 mm. In some other embodiments, the length L is comprised between about 300 mm and about 350 mm. In yet some other embodiments, the length L is comprised between about 310 mm and about 330 mm.

In some embodiments, the width W of the plagiocephaly pillow assembly 102 is comprised between about 100 mm and about 500 mm. In some other embodiments, the width W of the plagiocephaly pillow assembly 102 is comprised between about 150 mm and about 350 mm. In some other embodiments, the width W is comprised between about 200 mm and about 300 mm. In yet some other embodiments, the width W is comprised between about 250 mm and about 270 mm.

In some embodiments, and depending on the dimensions (in particular on a thickness) and number of the superposed resilient layers, the height H of the plagiocephaly pillow assembly 102 is comprised between about 10 mm and about 50 mm. In some other embodiments, the height H is comprised between about 20 mm and about 40 mm. In yet some other embodiments, the height H is comprised between about 35 mm and about 40 mm (i.e. comprised between about 1.38 in and about 1.57 in).

As represented in FIG. 8, the plagiocephaly pillow assembly 102 has first and second lateral edges 24, 26 extending substantially parallel to each other, and top and bottom edges 28, 30 spaced apart from each other and arranged between the first and second lateral edges 24, 26. The terms “top” and “bottom” should be understood with respect to a longitudinal direction defined by a body of the infant having their head at least partially supported by the plagiocephaly pillow assembly 102, i.e. bottom is intended to mean towards a torso of the infant, while top is intended to mean adjacent to a top of the infant head.

Shoulder-receiving recesses 32, 32′, 34, 34′ are formed respectively in the top and bottom edges 28, 30 that are dimensioned to cooperate and receive at least partially shoulders of the infant at least partially supported by the plagiocephaly pillow assembly 102, depending on an orientation of the plagiocephaly pillow assembly 102, in the embodiment wherein the plagiocephaly pillow assembly 102 is reversible. In other words, first and second shoulder-receiving recesses 34, 34′ are formed in the bottom edge 30 of the plagiocephaly pillow assembly 102, and third and fourth shoulder-receiving recesses 32, 32′ are formed in the top edge 28 of the plagiocephaly pillow assembly 102 (the top edge 28 forming a bottom edge of the plagiocephaly pillow assembly 102 configured in a reverse configuration). In the embodiment shown, the shoulder-receiving recesses 32, 32′, 34, 34′ are substantially curved, central bump portions 36, 38 being formed respectively between the shoulder-receiving recesses 32, 32′, 34, 34′ of the top and bottom edges 28, 30. The central bump portions 36, 38 are configured to cooperate with a portion of a neck of the infant.

The dimensions of at least one of the first and second shoulder-receiving recesses 34, 34′ formed in the bottom edge 30 could differ from the dimensions of at least one of the third and fourth shoulder-receiving recesses 32, 32′ formed in the top edge 28, so that the plagiocephaly pillow assembly 102 could be reversed so as to be adapted to infants having different morphologies. As a result, depending on which one of the top and bottom edges 28, 30 is placed against the shoulders of the infant, the plagiocephaly pillow assembly 102 could be adapted to different ages of the infant.

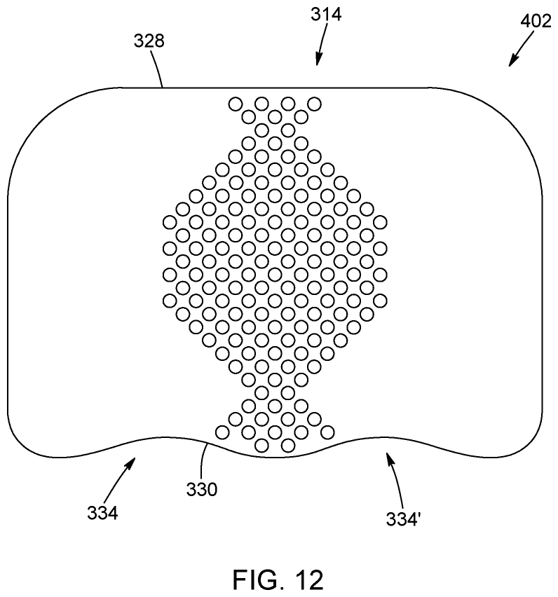

It is appreciated that, in alternative embodiments, the shape of the edges 24, 26, 28, 30 could differ from the embodiment shown. For instance and without being limitative, the top and bottom edges 28, 30 could be straight edges. In another embodiment, as represented in FIG. 12, the top edge 328 of the plagiocephaly pillow assembly 402 (for instance and without being limitative, of the second resilient layer 314, as shown in FIG. 12, but it is understood that the same remarks with regards to the top and bottom edges could also apply to the other resilient layers of the plagiocephaly pillow assembly 402) could be straight, whereas the bottom edge 330 could have shoulder-receiving recesses 334, 334′ formed therein.

Going back to FIGS. 1 to 10C, it is understood that the different resilient layers 10, 12, 14 are dimensioned to be superposed onto each other and to be received in the pillowcase 16 so as to form together the plagiocephaly pillow assembly 102. Moreover, in the embodiment shown, the different resilient layers 10, 12, 14 have substantially similar lengths and widths—considered in a substantially horizontal plane and corresponding substantially to the length L and the width W of the plagiocephaly pillow assembly 102—so that the terms lateral, top and bottom edges will indifferently be used with regards to the plagiocephaly pillow assembly 102, to the pillowcase 16 or to any one of the different superposable resilient layers 10, 12, 14. Moreover, it is understood that, in the embodiment shown, when the different resilient layer 10, 12, 14 are superposed onto each other, their respective top, bottom and first and second lateral edges are substantially vertically aligned with each other. It could however be conceived a plagiocephaly pillow assembly in which the different resilient layers would not have similar dimensions (for instance dimensions decreasing upwardly along the height of the plagiocephaly pillow assembly) so that at least some of the respective edges of the superposed resilient layers would be horizontally offset with regards to each other.

Pillowcase

The pillowcase 16 is configured to receive the different resilient layers 10, 12, 14 in the superposed configuration. In the embodiment shown, as represented in FIG. 2, an opening 40 is formed in one of the top and bottom edges 28, 30 of the pillowcase 16, for the resilient layers 10, 12, 14 to be easily introduced into and removed from the pillowcase 16. The pillowcase 16 further comprises fastener(s) 42, for instance a zipper, mounted adjacent to the opening 40, for the pillowcase 16 to be configured from a closed configuration, as represented in FIG. 3, to an open configuration, as represented in FIG. 2.

Moreover, as represented in FIG. 8, the pillowcase 16 has a bottom surface 35, configured to face a support surface when the plagiocephaly pillow assembly 102 is used and an opposed top surface. The pillowcase 16 can further comprise at least one support-fixing portion 37 (or at least one support-fixing portion, in the embodiment shown, for instance and without being limitative, two support-fixing strips 37 extending substantially parallel to each other and comprising a hook and loop fastening system) to contribute to the stability of the plagiocephaly pillow assembly 102 when it is supported on the support surface.

The pillowcase 16 is for instance at least partially made of fabrics, terry toweling or any soft material that could easily be washed.

Base Resilient Layer

It is understood that the base resilient layer 10 might be optional (i.e. the plagiocephaly pillow assembly 102 might be used only with the first and second resilient layers 12, 14), and for instance can be added into the pillowcase 16 when the plagiocephaly pillow assembly 102 is to be used with infants being older than a few months and/or which head is heavier than a pre-determined threshold.

In the embodiment shown, the base resilient layer 10 is at least partially made of a resilient material, such as a foam (for instance an open cell foam), a polyurethane foam or a visco-elastic foam.

In some embodiments, the base resilient layer 10 has a density comprised between about 0.5 pound per cubic foot and about 5 pounds per cubic foot. In some other embodiments, the density of the base resilient layer 10 is comprised between about 1 pound per cubic foot and about 3 pounds per cubic foot. In some other embodiments, the density of the base resilient layer 10 is comprised between about 1.1 pounds per cubic foot and about 1.5 pounds per cubic foot.

In some embodiments, the base resilient layer 10 has an Indentation Load Deflection (ILD) comprised between about 10 pounds and about 60 pounds. In some other embodiments, the ILD of the base resilient layer 10 is comprised between about 25 pounds and about 40 pounds. In some other embodiments, the ILD of the base resilient layer 10 is comprised between about 34 pounds and about 38 pounds.

It is to be noted that the ILD is measured by indenting (or compressing) a foam sample (a sample of the base resilient layer 10, in the present case) having a height of 4 in to 25 percent of its original height, using a disk having a diameter of 12 in. The amount of force (expressed in pounds) that is required to indent the foam (or the sample of the base resilient layer) to 25 percent of its original height corresponds to a measure of the ILD.

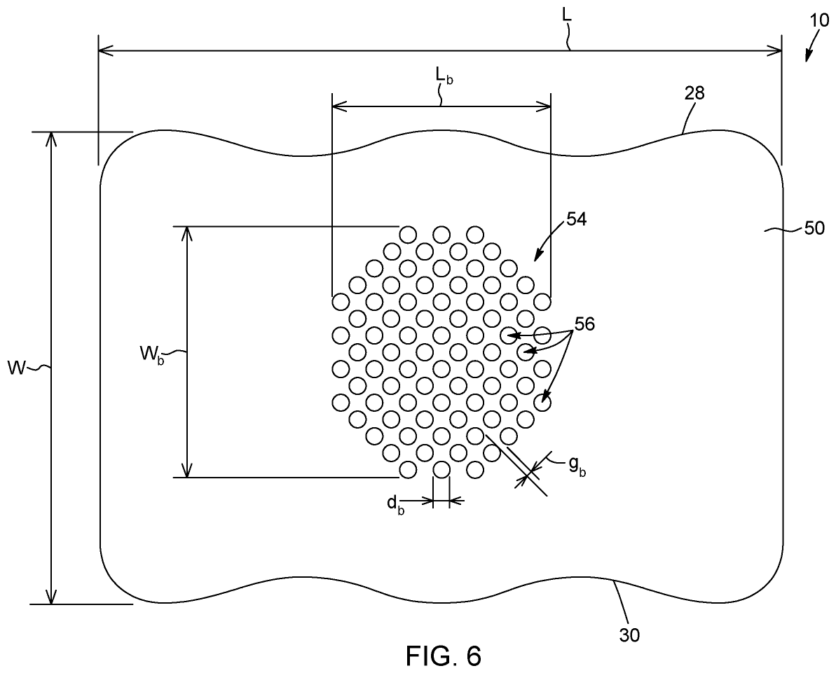

As represented in FIGS. 2 and 6, the base resilient layer 10 has a top surface 50 and an opposed bottom surface 52. The base resilient layer 10 has a set of base layer depressions 54—or base set of depressions 54—extending at least partially between the top and bottom surfaces 50, 52.

In some embodiments, the base resilient layer 10 has a thickness, considered along a substantially vertical direction and defined between the top and bottom surfaces 50, 52, comprised between about ⅛ in and about ⅞ in. In some other embodiments, the thickness of the base resilient layer 10 is comprised between about ⅜ in and about ⅝ in. In some other embodiments, the thickness of the base resilient layer 10 is comprised between about 7/16 in and about 9/16 in.

In the embodiment shown, as represented in FIG. 6, the base set of depressions 54 is formed substantially centrally in the base resilient layer 10 and has a substantially polygonal peripheral profile (for instance a substantially octagonal peripheral profile (or shape)).

The base set of depressions 54 has a length Lb, a width Wb and a base surface area. In the embodiment shown, the base set of depressions 54 is arranged so that the base layer 10 presents substantially horizontal and vertical axes of symmetry (or longitudinal and lateral axes of symmetry). Moreover, in the embodiment shown, the base set of depressions 54 is arranged within the base layer 10 so that the base layer 10 further presents a substantially horizontal plane of symmetry extending between the top and bottom surfaces 50, 52 of the base resilient layer 10.

In some embodiments, the length Lb is at least about 10% of the length L of the base layer 10. In some other embodiments, the length Lb is at least about 20% of the length L of the base layer 10. In some other embodiments, the length Lb is at least about 30% of the length L of the base layer 10. In yet some other embodiments, the length Lb is at least about 50% of the length L of the base resilient layer 10.

In some embodiments, the width Wb is at least about 20% of the width W of the base layer 10. In some other embodiments, the width Wb is at least about 40% of the width W of the base layer 10. In some other embodiments, the width Wb is at least about 50% of the width W of the base layer 10. In yet some other embodiments, the width Wb is at least about 70% of the width W of the base layer 10.

In some embodiments, the base surface area of the base set of depressions 54 is comprised between about 4 square in and about 20 square in. In some other embodiments, the base surface area of the set of base layer depressions 54 is comprised between about 7 square in and about 14 square in. In yet some other embodiments, the base surface area of the set of base layer depressions 54 is comprised between about 11 square in and about 13 square in.

In some embodiments, the base surface area of the base set of depressions 54 is bigger than about 3% of a surface area of the plagiocephaly pillow assembly 102. In some other embodiments, the base surface area of the base set of depressions 54 is bigger than about 5% of the surface area of the plagiocephaly pillow assembly 102. In some other embodiments, the base surface area of the base set of depressions 54 is bigger than about 7% of the surface area of the plagiocephaly pillow assembly 102. In yet some other embodiments, the base surface area of the base set of depressions 54 is bigger than about 9% of the surface area of the plagiocephaly pillow assembly 102.

In some embodiments, at least some of the depressions 54 extend along at least about 30% of the thickness of the base layer 10. In some other embodiments, at least some of the depressions 54 extend along at least about 50% of the thickness of the base layer 10. In yet some other embodiments, at least some of the depressions 54 extend along at least about 75% of the thickness of the base layer 10. In the embodiment shown, the set of base layer depressions 54 comprises at least some spaced-apart through holes 56 extending substantially through an entirety of the thickness of the base layer 10.

In the embodiment shown, at least some of the depressions 54 (for instance at least some of the through holes) have a substantially cylindrical shape defining a base diameter db.

In some embodiments, the base diameter db is comprised between about 1/32 in and about 31/32 in. In some other embodiments, the base diameter db is comprised between about 1/16 in and about 13/32 in. In some other embodiments, the base diameter db is comprised between 3/16 in and about 5/16 in. In the non-limitative embodiment shown, all the depressions of the set of base layer depressions 54 have substantially identical shapes and dimensions, but the base layer 10 might also comprise depressions having different dimensions and/or shapes.

In the non-limitative embodiment shown, the depressions of the set of base layer depressions 54 are regularly spaced apart from each other from a base gap distance gb. In some embodiments, the base gap distance gb is comprised between about 1/16 in and about 15/16 in. In some other embodiments, the base gap distance gb is comprised between about 1/16 in and about ⅜ in. In some other embodiments, the base gap distance gb is comprised between about 1/16 in and about 3/16 in.

It is appreciated that the shape and the dimensions of the base layer 10, as well as the shape, dimensions and locations of the depressions of the set of base layer depressions 54 can vary from the embodiment shown.

First Resilient Layer—Intermediate or Median Layer

In the embodiment shown, the first resilient layer 12 (or intermediate/median layer) is at least partially made of a resilient material, such as a foam (for instance an open cell foam), a polyurethane foam, a visco-elastic foam or a super soft foam.

In some embodiments, the first resilient layer 12 has a density comprised between about 0.5 pound per cubic foot and about 4 pounds per cubic foot. In some other embodiments, the density of the first resilient layer 12 is comprised between about 1 pound per cubic foot and about 2 pounds per cubic foot. In yet some other embodiments, the density of the first resilient layer 12 is comprised between about 1.1 pounds per cubic foot and about 1.3 pounds per cubic foot.

In some embodiments, the first resilient layer 12 has an Indentation Load Deflection (ILD) comprised between about 8 pounds and about 25 pounds. In some other embodiments, the ILD of the first resilient layer 12 is comprised between about 12 pounds and about 20 pounds. In some other embodiments, the ILD of the first resilient layer 12 is comprised between about 10 pounds and about 14 pounds.

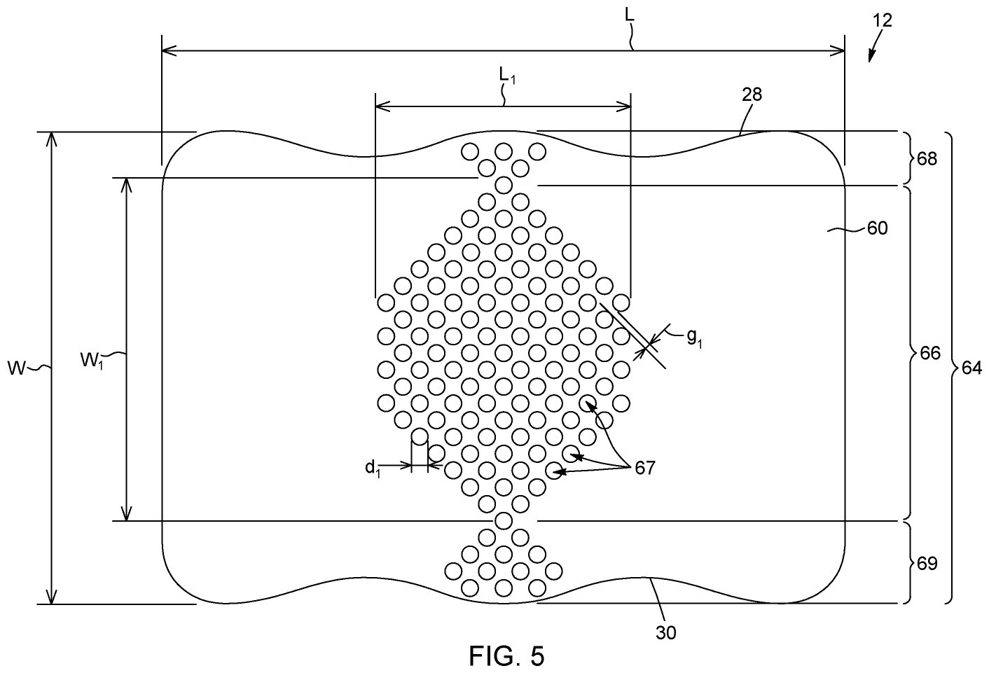

As represented in FIGS. 2 and 5, the first resilient layer 12 has a top surface 60 and an opposed bottom surface 62. When the first resilient layer 12 is superposed to the base layer 10, as represented in FIG. 2, the bottom surface 62 of the first resilient layer 12 at least partially faces and is at least partially abutted against the top surface 50 of the base resilient layer 10.

The first resilient layer 12 has a first set of depressions 64 extending at least partially between the top and bottom surfaces 60, 62. In the embodiment shown, the first set of depressions 64 is arranged so that the first resilient layer 12 presents substantially horizontal and vertical axes of symmetry (i.e. longitudinal and lateral axes of symmetry). Moreover, in the embodiment shown, the first set of depressions 64 is arranged within the first resilient layer 12 so that the first resilient layer 12 further presents a substantially horizontal plane of symmetry extending between the top and bottom surfaces 60, 62.

In some embodiments, the first resilient layer 12 has a thickness, considered along a substantially vertical direction and defined between the top and bottom surfaces 60, 62, comprised between about ⅛ in and about ⅞ in. In some other embodiments, the thickness of the first resilient layer 12 is comprised between about ⅜ in and about 9/16 in. In yet some other embodiments, the thickness of the first resilient layer 12 is comprised between about 7/16 in and about 9/16 in.

In the embodiment shown, the first set of depressions 64 is formed substantially centrally in the first resilient layer 12 and comprises a central portion 66 having a substantially polygonal peripheral profile (for instance a substantially hexagonal peripheral profile (or shape)), located between a top portion 68 and a bottom portion 69. The top and bottom portions 68, 69 of the first set of depressions 64 are located respectively in the vicinity of the top and bottom edges 28, 30 of the first resilient layer 12.

The central portion 66 of the first set of depressions 54 has a length L1 and a width W1.

In some embodiments, the length L1 is at least about 10% of the length L of the first layer 12. In some other embodiments, the length L1 is at least about 20% of the length L of the first layer 12. In some other embodiments, the length L1 is at least about 30% of the length L of the first layer 12. In yet some other embodiments, the length L1 is at least about 50% of the length L of the first layer 12.

In some embodiments, the width W1 is at least about 30% of the width W of the first layer 12. In some other embodiments, the width W1 is at least about 50% of the width W of the first layer 12. In some other embodiments, the width W1 is at least about 65% of the width W of the first layer 12. In yet some other embodiments, the width W1 is at least about 75% of the width W of the first layer 12.

In the embodiment shown, the top and bottom portion 68, 69 have a substantially polygonal shape (for instance a substantially triangular or trapezoidal shape) and are dimensioned so that the first set of depressions 64 comprising the top and bottom portions 68, 69 and the central portion 66 extends substantially along an entirety of the width W of the first layer 12.

The first set of depressions 64 has a first surface area. In some embodiments, the first surface area of the first set of depressions 64 is comprised between about 8 square in and about 30 square in. In some other embodiments, the first surface area of the first set of depressions 64 is comprised between about 12 square in and about 25 square in. In yet some other embodiments, the first surface area of the first set of depressions 64 is comprised between about 15 square in and about 17 square in.

In some embodiments, the first surface area of the first set of depressions 64 is bigger than about 5% of a surface area of the plagiocephaly pillow assembly 102. In some other embodiments, the first surface area of the first set of depressions 64 is bigger than about 8% of the surface area of the plagiocephaly pillow assembly 102. In some other embodiments, the first surface area of the first set of depressions 64 is bigger than about 10% of the surface area of the plagiocephaly pillow assembly 102. In yet some other embodiments, the first surface area of the first set of depressions 64 is bigger than about 12% of the surface area of the plagiocephaly pillow assembly 102.

In some embodiments, at least some of the depressions 64 extend along at least about 30% of the thickness of the first resilient layer 12. In some other embodiments, at least some of the depressions 64 extend along at least about 50% of the thickness of the first resilient layer 12. In yet some other embodiments, at least some of the depressions 64 extend along at least about 75% of the thickness of the first resilient layer 12. In the embodiment shown, the first set of depressions 64 comprises at least some through holes 67 extending substantially along an entirety of the thickness of the first layer 12.

In the embodiment shown, at least some of the depressions 64—for instance at least some of the through holes—have a substantially cylindrical shape defining a first diameter d1. In some embodiments, the first diameter d1 is comprised between about 1/32 in and about 31/32 in. In some other embodiments, the first diameter d1 is comprised between about 1/16 in and about 13/32 in. In yet some other embodiments, the first diameter d1 is comprised between 3/16 in and about 5/16 in.

In the non-limitative embodiment shown, all the depressions of the first set of depressions 64 have substantially identical shapes and dimensions, but the first layer 12 might also comprise depressions having different dimensions and/or shapes.

In the non-limitative embodiment shown, the depressions of the first set of depressions 64 are regularly spaced apart from each other from a first gap distance g1. In some embodiments, the first gap distance g1 is comprised between about 1/16 in and about 15/16 in. In some other embodiments, the first gap distance g1 is comprised between about 1/16 in and about ⅜ in. In yet some other embodiments, the first gap distance g1 is comprised between about 1/16 in and about 3/16 in.

It is appreciated that the shape and the dimensions of the first (or intermediate/median) layer 12, as well as the shape, dimensions and locations of the depressions of the first set of depressions 64 can vary from the embodiment shown.

Second Resilient Layer—Top Layer

In the embodiment shown, the second resilient layer 14 (or top resilient layer 14) is at least partially made of a resilient material, such as a foam (for instance an open cell foam), a polyurethane foam, a visco-elastic foam or a super soft foam.

In some embodiments, the second resilient layer 14 has a density comprised between about 0.5 pound per cubic foot and about 4 pounds per cubic foot. In some other embodiments, the density of the second resilient layer 14 is comprised between about 1 pound per cubic foot and about 2 pounds per cubic foot. In yet some other embodiments, the density of the second resilient layer 14 is comprised between about 1.1 pounds per cubic foot and about 1.3 pounds per cubic foot.

In some embodiments, the second resilient layer 14 has an Indentation Load Deflection (ILD) comprised between about 8 pounds and about 20 pounds. In some other embodiments, the ILD of the second resilient layer 14 is comprised between about 10 pounds and about 15 pounds. In yet some other embodiments, the ILD of the second resilient layer 14 is comprised between about 11 pounds and about 13 pounds.

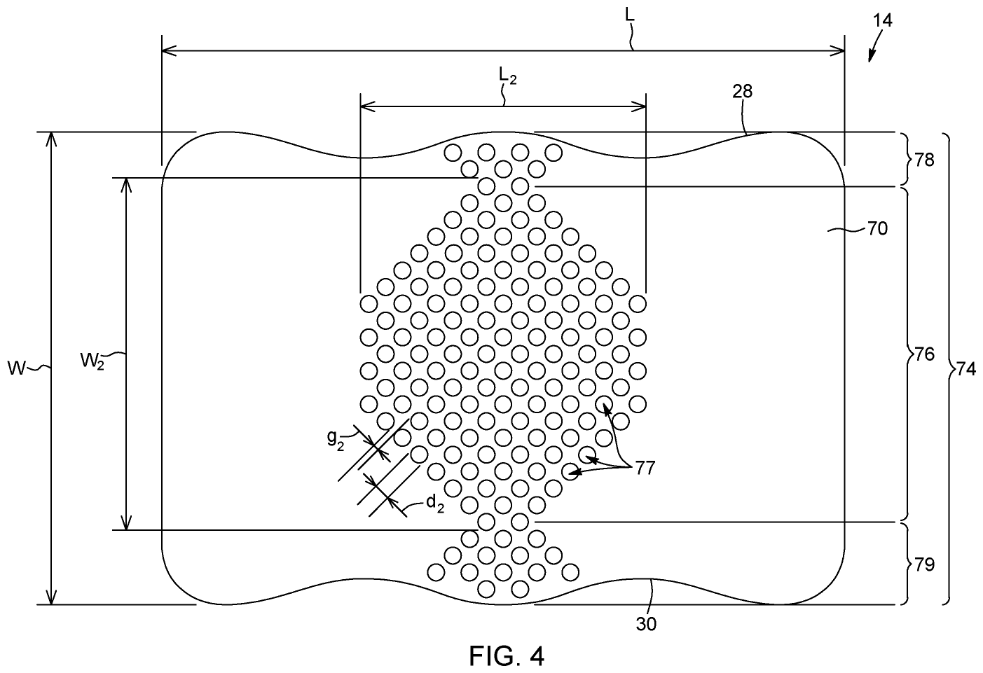

As represented in FIGS. 2 and 4, the second resilient layer 14 has a top surface 70 and an opposed bottom surface 72. When the second resilient layer 14 is superposed to the first resilient layer 12, as represented in FIG. 2, the bottom surface 72 of the second resilient layer 14 at least partially faces and is at least partially abutted against the top surface 60 of the first resilient layer 12. The top surface 70 of the second resilient layer 14 is configured to receive and support at least a portion of the head of the infant.

The second resilient layer 14 has a second set of depressions 74 extending at least partially between the top and bottom surfaces 70, 72. In the embodiment shown, the second set of depressions 74 is arranged so that the second resilient layer 14 presents substantially horizontal and vertical axes of symmetry (or longitudinal and lateral axes of symmetry). Moreover, in the embodiment shown, the second set of depressions 74 is arranged within the second resilient layer 14 so that the second resilient layer 14 further presents a substantially horizontal plane of symmetry extending between the top and bottom surfaces 70, 72.

In some embodiments, the second resilient layer 14 has a thickness, considered along a substantially vertical direction and defined between the top and bottom surfaces 70, 72, comprised between about ⅛ in and about ⅞ in. In some other embodiments, the thickness of the second resilient layer 14 is comprised between about ⅜ in and about 9/16 in. In yet some other embodiments, the thickness of the second resilient layer 14 is comprised between about 7/16 in and about 9/16 in.

In the embodiment shown, the second set of depressions 74 is formed substantially centrally in the second resilient layer 14 and comprises a central portion 76 having a substantially polygonal shape (for instance a substantially hexagonal shape), located between a top portion 78 and a bottom portion 79. The top and bottom portions 78, 79 of the second set of depressions 74 are located respectively in the vicinity of the top and bottom edges 28, 30 of the second resilient layer 14.

The central portion 76 of the second set of depressions 64 has a length L2 and a width W2.

In some embodiments, the length L2 is at least about 10% of the length L of the second layer 14. In some other embodiments, the length L2 is at least about 25% of the length L of the second layer 14. In some other embodiments, the length L2 is at least about 40% of the length L of the second layer 14. In yet some other embodiments, the length L2 is at least about 60% of the length L of the second resilient layer 14.

In some embodiments, the width W2 is at least about 30% of the width W of the second layer 14. In some other embodiments, the width W2 is at least about 60% of the width W of the second layer 14. In some other embodiments, the width W2 is at least about 70% of the width W of the second layer 14. In yet some other embodiments, the width W2 is at least about 85% of the width W of the second layer 14.

In the embodiment shown, the top and bottom portions 78, 79 have a substantially polygonal shape (for instance a substantially triangular or trapezoidal shape) and are dimensioned so that the second set of depressions 74, comprising the top and bottom portions 78, 79 and the central portion 76, extends substantially along the entire width W of the second layer 14.

The second set of depressions 74 has a second surface area. In some embodiments, the second surface area is comprised between about 15 square in and about 40 square in. In some other embodiments, the second surface area is comprised between about 15 square in and about 32 square in. In yet some other embodiments, the second surface area is comprised between about 16 square in and about 20 square in.

In some embodiments, the second surface area of the second set of depressions 74 is bigger than about 5% of a surface area of the plagiocephaly pillow assembly 102. In some other embodiments, the second surface area of the second set of depressions 74 is bigger than about 10% of the surface area of the plagiocephaly pillow assembly 102. In some other embodiments, the second surface area of the second set of depressions 74 is bigger than about 12% of the surface area of the plagiocephaly pillow assembly 102. In yet some other embodiments, the second surface area of the second set of depressions 74 is bigger than about 14% of the surface area of the plagiocephaly pillow assembly 102.

In some embodiments, at least some of the depressions 74 extend along at least about 30% of the thickness of the second resilient layer 14. In some other embodiments, at least some of the depressions 74 extend along at least about 50% of the thickness of the second resilient layer 14. In yet some other embodiments, at least some of the depressions 74 extend along at least about 75% of the thickness of the second resilient layer 14. In the embodiment shown, the second set of depressions 74 comprises at least some through holes 77 extending substantially along an entirety of the thickness of the second layer 14.

In the embodiment shown, at least some of the depressions 74—for instance at least some of the through holes—have a substantially cylindrical shape defining a second diameter d2. In some embodiments, the second diameter d2 is comprised between about 1/32 in and about 31/32 in. In some other embodiments, the second diameter d2 is comprised between about 1/16 in and about 13/32 in. In yet some other embodiments, the second diameter d2 is comprised between 3/16 in and about 5/16 in.

In the non-limitative embodiment shown, all the depressions of the second set of depressions 74 have substantially identical shapes and dimensions, but the second layer 14 might also comprise depressions having different dimensions and/or shapes.

In the non-limitative embodiment shown, the depressions of the second set of depressions 74 are regularly spaced apart from each other from a second gap distance g2. In some embodiments, the second gap distance g2 is comprised between about 1/16 in and about 15/16 in. In some other embodiments, the second gap distance g2 is comprised between about 1/16 in and about ⅜ in. In yet some other embodiments, the second gap distance g2 is comprised between about 1/16 in and about 3/16 in.

It is appreciated that the shape and the dimensions of the second (or top) layer 14, as well as the shape, dimensions and locations of the depressions of the second set of depressions 74 can vary from the embodiment shown.

Relative Features of the Different Layers

The plagiocephaly pillow assembly 102 is configured so that at least some of the depressions of one of the first and second sets of depressions 64, 74 are at least partially offset and/or shifted (considered in a substantially horizontal plane) with regards to the depressions of the other one of the first and second sets of depressions 64, 74 when the first and second resilient layers 12, 14 are superposed onto each other. The offset and/or the shifting of the depressions of the first and second resilient layers 12, 14 should be understood in a projection plane extending substantially transversally to a direction of the depressions (i.e. substantially transversally to the thickness of the first and/or second resilient layers 12, 14) (for instance in a projection plane extending substantially horizontally or parallel to the top surfaces 60, 70 and the bottom surfaces 62, 72 of the first and second resilient layers 12, 14).

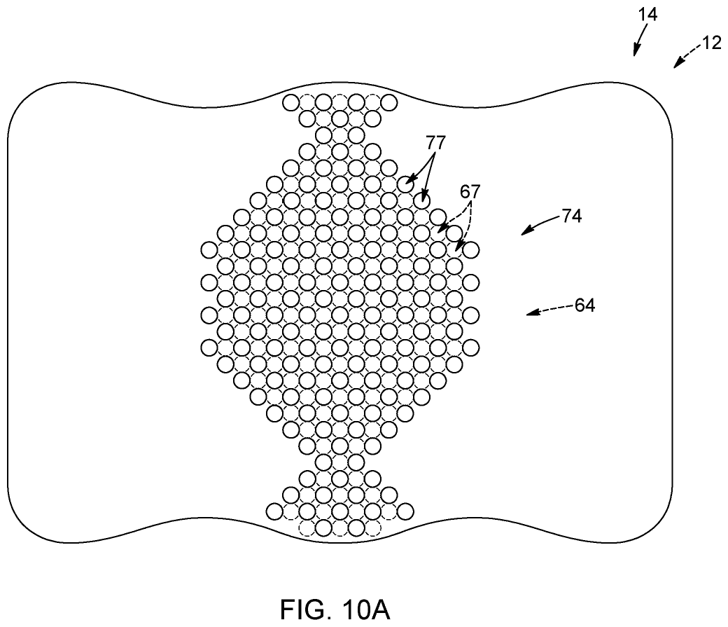

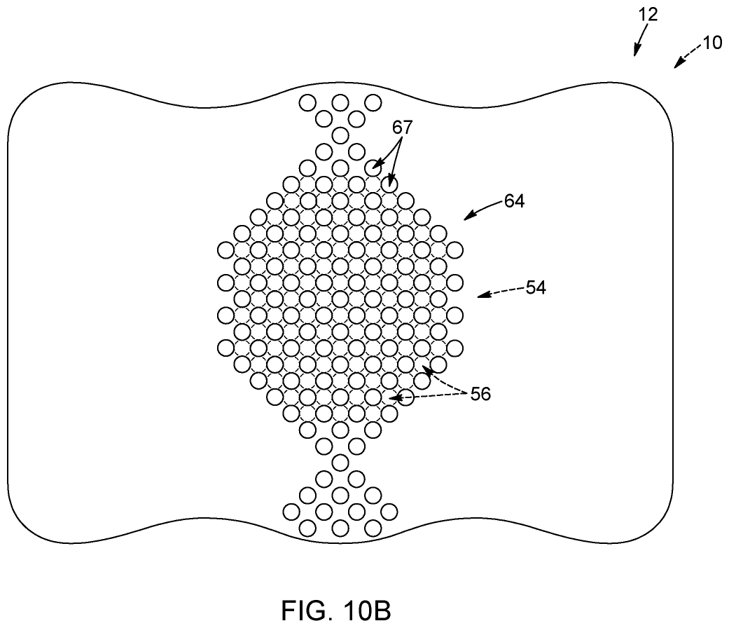

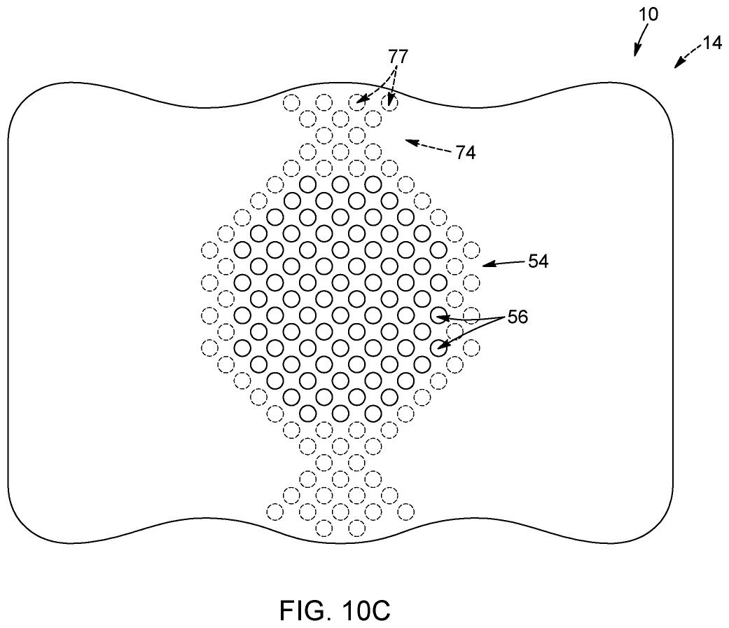

In other words, and as represented in FIG. 10A in which the first set of depressions is represented in dotted lines, the first and second sets of depressions 64, 74 comprising the through holes 67, 77 are arranged so that, considered projected in a plane substantially parallel to at least one of the top and bottom surfaces of the first and second resilient layers 12, 14 (for instance in a plane substantially horizontal), at least some of the depressions of one of the first and second sets of depressions 64, 74 are not in register or aligned, i.e. they are at least partially offset, with any of the depressions of the other of the first and second sets of depressions 64, 74 when the first and second resilient layers 12, 14 are superposed onto each other (for instance when at least some of the edges of the first and second resilient layers 12, 14 are substantially vertically aligned with each other).

In yet other words, considered projected in a plane substantially parallel to at least one of the top and bottom surfaces of the first and second resilient layers 12, 14, at least some of the depressions of one of the first and second sets of depressions 64, 74 extend (or are arranged) at least partially between some of the depressions of the other of the first and second sets of depressions 64, 74 when the first and second layers 12, 14 are superposed onto each other in a predetermined configuration, which can be a configuration wherein the peripheral edges of the first and second layers 12, 14 are in register.

It is thus understood that the first and second sets of depressions 64, 74 define respectively first and second depression patterns having a width, a length and a surface area, the first and second depression patterns distinguishing from each other by at least one of the numbers, the shapes, the dimensions, the locations of at least some of their respective depressions, and the width, the length and the surface area of their respective depression patterns.

The plagiocephaly pillow assembly 102 is further configured so that at least some of the depressions of the base set of depressions 54 are at least partially offset and/or shifted with regards to the depressions of at least one of the first and second sets of depressions 64, 74 when the first and second resilient layers 12, 14 and the base resilient layer 10 are superposed on each other. The offset and/or the shifting of the depressions of the base layer 10 with at least one of the first and second resilient layers 12, 14 should be understood in a projection plane extending substantially transversally to the thickness of the base, first and/or second resilient layers 10, 12, 14 or, as mentioned above, parallel to the top surfaces 50, 60, 70 and the bottom surfaces 52, 62, 72 of the base, first and second resilient layers 10, 12, 14.

In other words, the base set of depressions 54 and the at least one of the first and second sets of depressions 64, 74 are arranged so that, considered projected in a plane substantially parallel to at least one of the top and bottom surfaces of the base, first and second resilient layers 10, 12, 14, at least some of the depressions of the base layer 10 are not in register or aligned with any of the depressions of the at least one of the first and second sets of depressions 64, 74 when the base, first and second resilient layers 10, 12, 14 are superposed onto each other (for instance when at least some of the edges of the base, first and second resilient layers 10, 12, 14 are substantially aligned with each other).

In yet other words, considered projected in a plane substantially parallel to at least one of the top and bottom surfaces of the base, first and second resilient layers 10, 12, 14, at least some of the depressions of the base set of depressions 54 extend (or are arranged) at least partially between some of the depressions of the at least one of the first and second sets of depressions 64, 74 when the base, first and second layers 12, 14 are superposed onto each other.

It is thus understood that the base set of depressions 54 define respectively a base depression pattern having a width, a length and a surface area, the base depression pattern distinguishing with the at least one of the first and second depression patterns by at least one of the numbers, the shapes, the dimensions, the locations of at least some of their respective depressions, and the width, the length and the surface area of their respective depression patterns.

In the embodiment shown, and as represented in FIGS. 10B and 10C in which the base set of depressions 54 comprising the through holes 56 and the second set of depressions 74 comprising the through holes 77 are respectively represented in dotted lines, at least some of the depressions formed in the base layer 10 are at least partially offset with regards to the depressions of the first set of depressions 64 comprising the through holes 67 and some of the depressions of the second set of depressions 74 are at least partially offset with regards to the depressions formed in the base layer 10.

In some embodiments, the surface area of the second set of depressions 74 (i.e. the second surface area) is greater than the surface area of the first set of depressions 64 (i.e. the first surface area). In some embodiments, the second surface area of the second set of depressions 74 is comprised between about 105% and about 210% of the first surface area of the first set of depressions 64. In some other embodiments, the second surface area of the second set of depressions 74 is comprised between about 110% and about 130% of the first surface area of the first set of depressions 64. In some other embodiments, the second surface area of the second set of depressions 74 is comprised between about 113% and about 128% of the first surface area of the first set of depressions 64. In yet some other embodiments, the second surface area of the second set of depressions 74 is comprised between about 111% and about 115% of the first surface area of the first set of depressions 64.

In some embodiments, the second surface area of the second set of depressions 74 is greater than the base surface area of the set of base layer depressions 54. In some embodiments, the second surface area of the second set of depressions 74 is comprised between about 110% and about 300% of the base surface area of the set of base layer depressions 54. In some other embodiments, the second surface area of the second set of depressions 74 is comprised between about 145% and about 230% of the base surface area of the set of base layer depressions 54. In some other embodiments, the second surface area of the second set of depressions 74 is comprised between about 150% and about 229% of the base surface area of the set of base layer depressions 54. In yet some other embodiments, the second surface area of the second set of depressions 74 is comprised between about 148% and about 152% of the base surface area of the set of base layer depressions 54.

In some embodiments, the first surface area of the first set of depressions 64 is greater than the base surface area of the set of base layer depressions 54. In some embodiments, the first surface area of the first set of depressions 64 is comprised between about 110% and about 300% of the base surface area of the set of base layer depressions 54. In some other embodiments, the first surface area of the first set of depressions 64 is comprised between about 145% and about 230% of the base surface area of the set of base layer depressions 54. In some other embodiments, the first surface area of the first set of depressions 64 is comprised between about 150% and about 229% of the base surface area of the set of base layer depressions 54. In yet some other embodiments, the first surface area of the first set of depressions 64 is comprised between about 148% and about 152% of the base surface area of the set of base layer depressions 54.

In some embodiments, the ILD of the second resilient layer 14 is smaller than the ILD of the first resilient layer 12. In some other embodiments, the ILD of the second resilient layer 14 is smaller than about 90% of the ILD of the first resilient layer 12. In some other embodiments, the ILD of the second resilient layer 14 is smaller than about 75% of the ILD of the first resilient layer 12. In yet some other embodiments, the ILD of the second resilient layer 14 is smaller than about 50% of the ILD of the first resilient layer 12.

In some embodiments, the ILD of at least one of the first and second resilient layers 12, 14 is smaller than the ILD of the base layer 10. In some other embodiments, the ILD of the at least one of the first and second resilient layers 12, 14 is smaller than about 90% of the ILD of the base layer 10. In some other embodiments, the ILD of the at least one of the first and second resilient layers 12, 14 is smaller than about 75% of the ILD of the base layer 10. In yet some other embodiments, the ILD of the at least one of the first and second resilient layers 12, 14 is smaller than about 50% of the ILD of the base layer 10.

In other words, in some embodiments, the plagiocephaly pillow assembly 102 has an Indentation Load Deflection (ILD) decreasing upwardly along the height H of the plagiocephaly pillow assembly 102 (i.e. an ILD decreasing between the bottom surface 52 of the base resilient layer 10 and the top surface 70 of the second resilient layer 14).

In some embodiments, the density of the second resilient layer 14 is smaller than the density of the first resilient layer 12. In some other embodiments, the density of the second resilient layer 14 is smaller than about 90% of the density of the first resilient layer 12. In some other embodiments, the density of the second resilient layer 14 is smaller than about 75% of the density of the first resilient layer 12. In yet some other embodiments, the density of the second resilient layer 14 is smaller than about 50% of the density of the first resilient layer 12.

In some embodiments, the density of at least one of the first and second resilient layers 12, 14 is smaller than the density of the base layer 10. In some other embodiments, the density of the at least one of the first and second resilient layers 12, 14 is smaller than about 90% of the density of the base layer 10. In some other embodiments, the density of the at least one of the first and second resilient layers 12, 14 is smaller than about 75% of the density of the base layer 10. In yet some other embodiments, the density of the at least one of the first and second resilient layers 12, 14 is smaller than about 50% of the density of the base layer 10.

In other words, in some embodiments, the plagiocephaly pillow assembly 102 has a density decreasing upwardly along the height H of the plagiocephaly pillow assembly (i.e. a density decreasing between the bottom surface 52 of the base resilient layer 10 and the top surface 70 of the second resilient layer 14).

The depressions are thus arranged in the first and second layers 12, 14 and in the base layer 10 so that the head of the infant is supported in a stable and comfortable manner by the plagiocephaly pillow assembly 102 when the infant is placed face-up on the plagiocephaly pillow assembly 102. Due to the particular arrangement of the depressions formed in the first and second layers 12, 14 and in the base layer 10, when the plagiocephaly pillow assembly 102 comprises the base layer 10, the pressure sustained by the head of the infant when the infant is laid face-up is better distributed with regard to pillows in which no depressions are formed and/or in which no depressions are formed in an offset manner between superposed layers, thus limiting the risk of plagiocephaly and/or brachycephaly.

It is understood that, in the embodiment (not represented) in which at least some of the superposed resilient layers would not have identical dimensions or identical peripheral profile, the plagiocephaly pillow assembly would be configurable into one or more superposed configuration wherein the different superposed resilient layers would be arranged so that at least some of the depressions of one of the sets of depressions would be at least partially offset with regards to the depressions of another one of the sets of depressions.

Moreover, even though in the embodiment shown, the first, second and base resilient layers are removably superposable onto each other, it could also be conceived a plagiocephaly pillow assembly in which at least some of the different layers would be permanently secured to each other (for instance by being glued or sewn to each other). It could also be conceived a plagiocephaly pillow assembly that would be formed by additive manufacturing so as to form different layers with different densities and/or ILD and/or with sets of depressions formed therein, wherein depression patterns of the different layers would differ from each other by at least one of a width, a length, a surface area thereof and a number, dimensions and locations of the depressions.

### Cushion

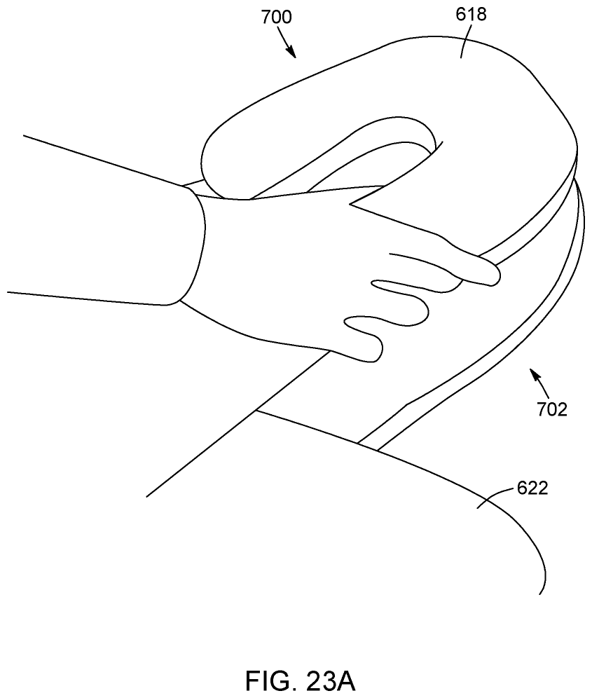

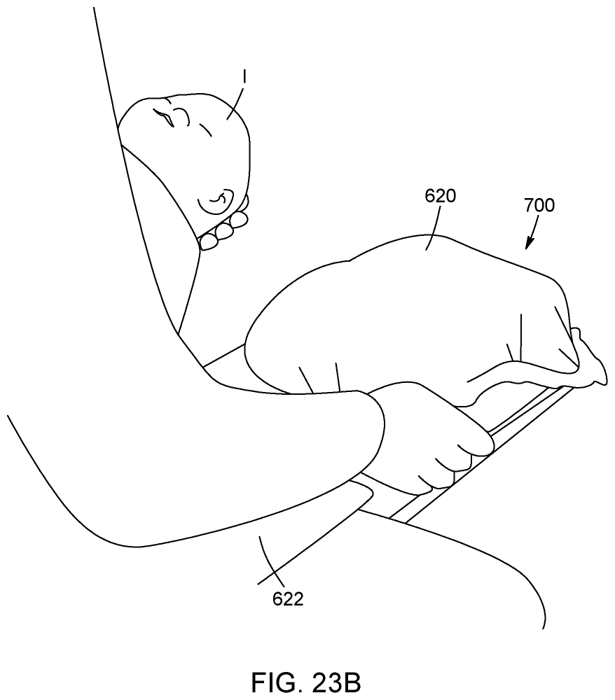

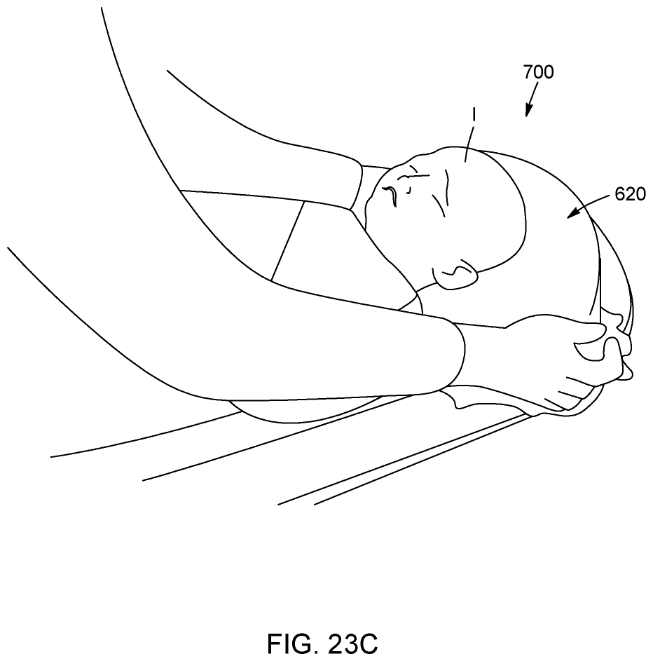

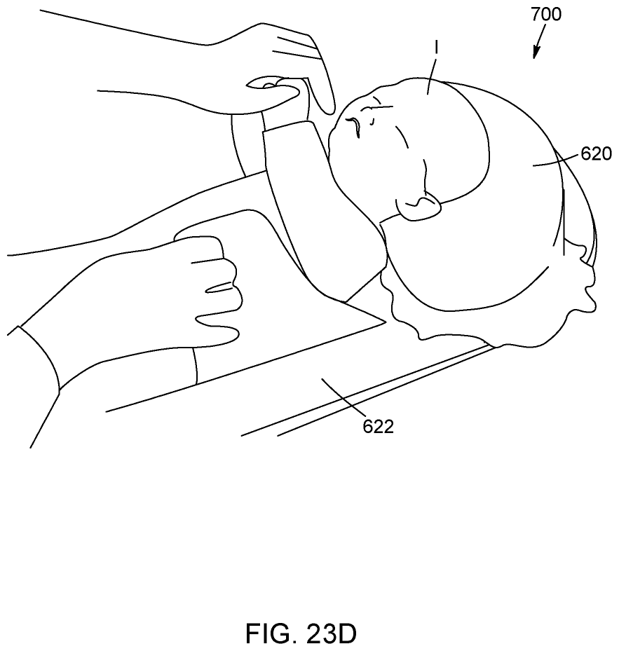

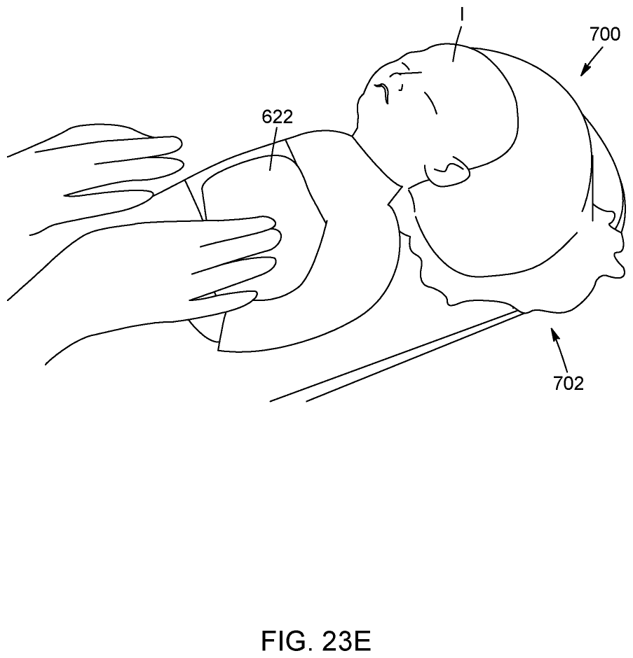

As represented in FIG. 7, and as it will be more apparent in FIG. 23A relative to another embodiment of the plagiocephaly prevention set, in the embodiment shown, the cushion 18 (or head-surrounding cushion 18) is configured to be arranged on the top surface of the second resilient layer (or on a top surface of the pillowcase 16, when the different layers are arranged in the pillowcase), so as to further limit the movements of the head of the infant when they are laid face-up on the plagiocephaly pillow assembly 102.

As represented in FIG. 7, in the embodiment shown, the cushion 18 has a substantially arcuated shape (or a substantially U shape), so as to better conform to the shape of the head of the infant. In some other embodiments, the cushion is at least partially deformable into a substantially C-shape cushion. The cushion 18 is thus shaped and dimensioned to at least partially surround the head of the infant (i.e. to extend along at least a portion of upper and/or lateral portions of the head of the infant).

It is appreciated that the shape, the structure and the configuration of the cushion 18 can vary from the embodiment shown. It could also be conceived a plagiocephaly prevention set that would not comprise a head-surrounding cushion.

### Protection Cover

The protection cover 20, as represented in FIG. 1, is dimensioned to at least partially cover the plagiocephaly pillow assembly 102, possibly when the cushion 18 is arranged on the top surface of the pillowcase 16 containing the different superposed resilient layers.

The protection cover 20 is for instance at least partially made of fabrics, terry toweling or any soft material that could easily be washed.

It is appreciated that the shape, the dimensions and the configuration of the protection cover 20 can vary from the embodiment shown. It could also be conceived a plagiocephaly prevention set that would not comprise a protection cover.

### Wrapping Cover

The wrapping cover 22 is dimensioned to at least partially wrap the infant once their head is supported on the plagiocephaly pillow assembly 102.

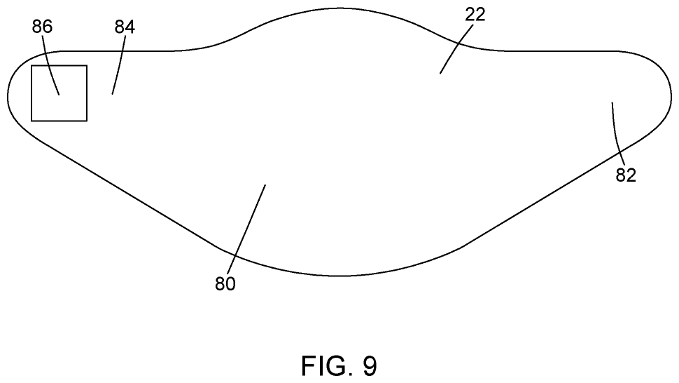

In the embodiment shown, as represented in FIGS. 1 and 9, the wrapping cover 22 comprises a body-receiving section 80, configured and dimensioned to receive at least a portion of the body of the infant (for instance a portion of their back), and two opposed wrapping sections 82, 84 configured to be folded on each other to wrap the infant.

In the embodiment shown, the wrapping cover 22 further comprises a fastener 86 at at least one of the wrapping sections 82, 84 (such as a hook and loop fastening system), to maintain the wrapping sections 82, 84 together when the infant is wrapped in the wrapping cover 22 and when the wrapping sections 82, 84 are folded on each other.

The wrapping cover 22 is for instance at least partially made of fabrics, terry toweling or any soft material that could easily be washed.

It is appreciated that the shape, the dimensions and the configuration of the wrapping cover 22 can vary from the embodiment shown. It could also be conceived a plagiocephaly prevention set that would not comprise a wrapping cover.

### Other Possible Embodiments

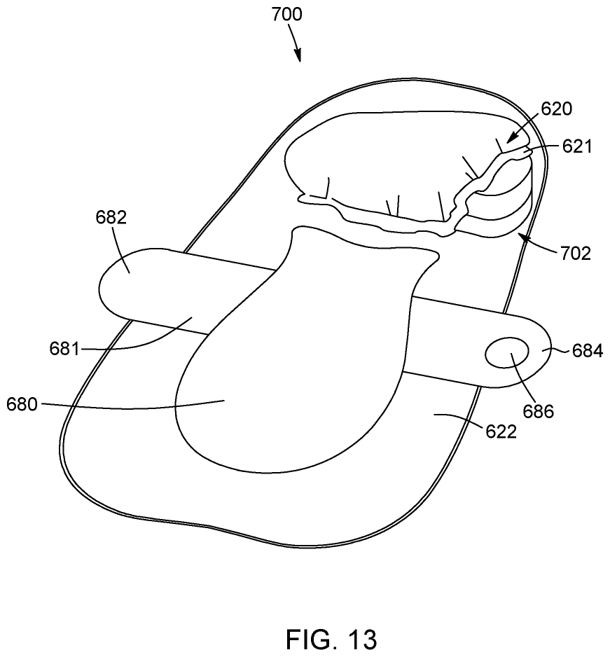

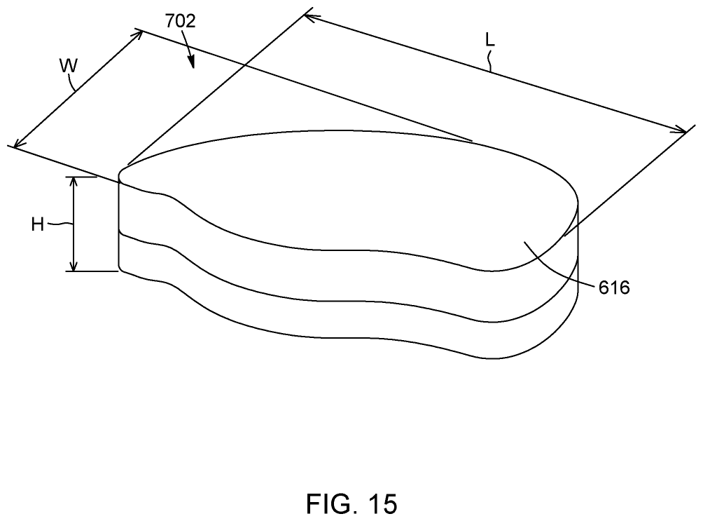

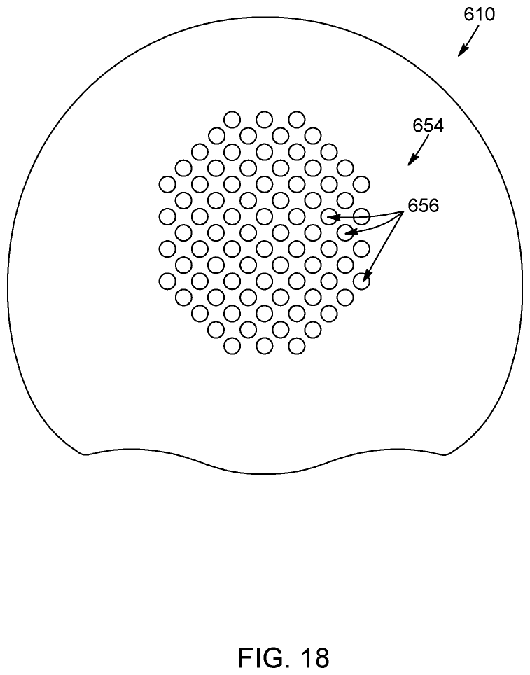

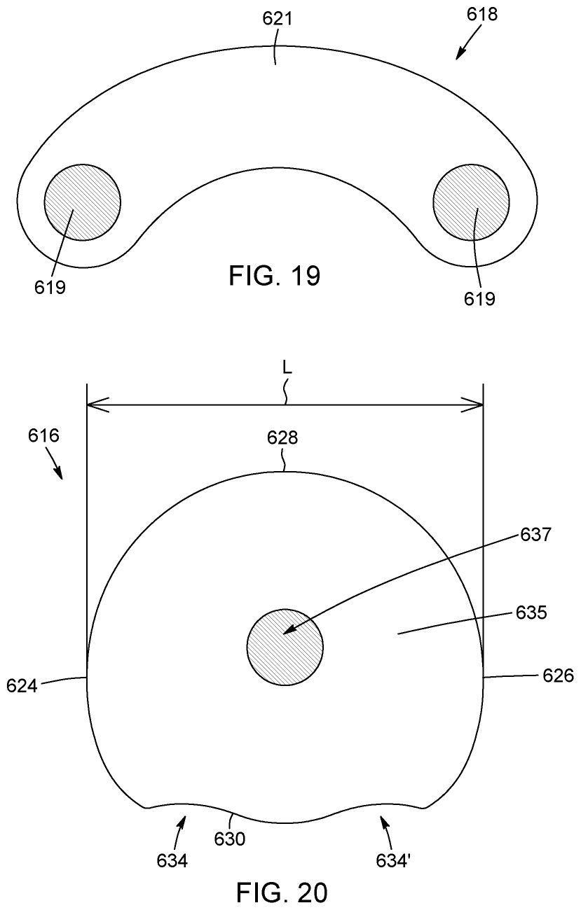

FIGS. 13 to 22C represent another embodiment of the plagiocephaly prevention set 700 according to the present disclosure.

The embodiment represented in FIGS. 13 to 22C of the plagiocephaly prevention set 700 distinguishes mainly from the embodiments of FIGS. 1 to 10C and FIG. 12 by the shape and dimensions of some of its elements.

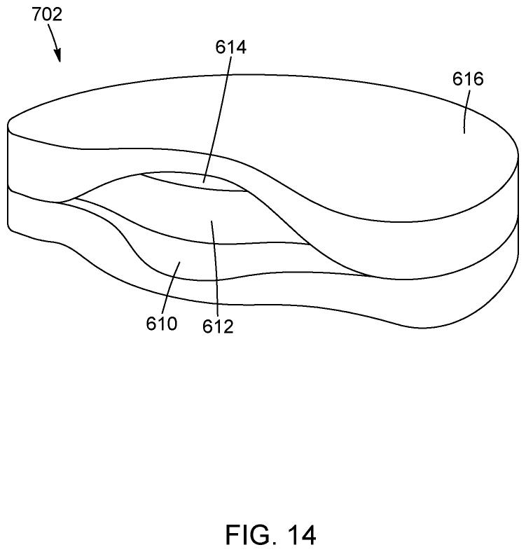

Firstly, as represented in FIGS. 14 to 18, the plagiocephaly pillow assembly 702 and its three superposed resilient layers 610, 612, 614 have a substantially circular shape, so that the width W of the plagiocephaly pillow assembly 702 corresponds substantially to its length L. In the embodiment shown, the first and second lateral edges 624, 626 (FIG. 20) and the top edge 628 of the plagiocephaly pillow assembly 702 form together a substantially continuous circular arc, whereas the bottom edge 630 forms a chord (comprising substantially rounded portions, in the embodiment shown) of the sector of the circle defined by the top, first and second lateral edges.

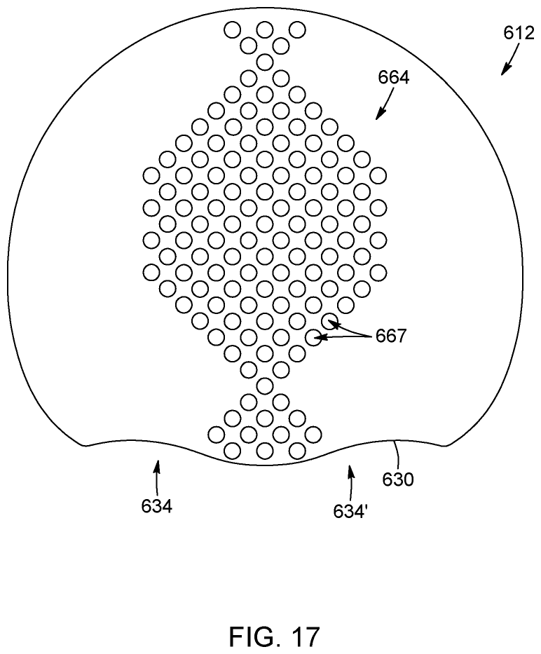

As represented in FIG. 17, the bottom edge 630 has shoulder-receiving recesses 634, 634′ formed therein, to cooperate and receive at least partially the shoulders of the infant at least partially supported by the plagiocephaly pillow assembly 702.

The pillowcase 616, as represented in FIGS. 14, 15 and 20, is thus also substantially circular in shape and has a support-fixing portion 37 (or support-fixing disk 637, in the embodiment shown) located substantially centrally on the bottom surface 635 to contribute to the stability of the plagiocephaly pillow assembly 702 when it is supported on the support surface.

Moreover, the protection cover 620, as represented in FIG. 13, is dimensioned to at least partially cover the plagiocephaly pillow assembly 702 and might be substantially mobcap-shaped. The protection cover 620 might further comprise a peripheral retainer sleeve 621, comprising for instance an elastic band, to better maintain the protection cover 620 onto (or at least partially around) the plagiocephaly pillow assembly 702.

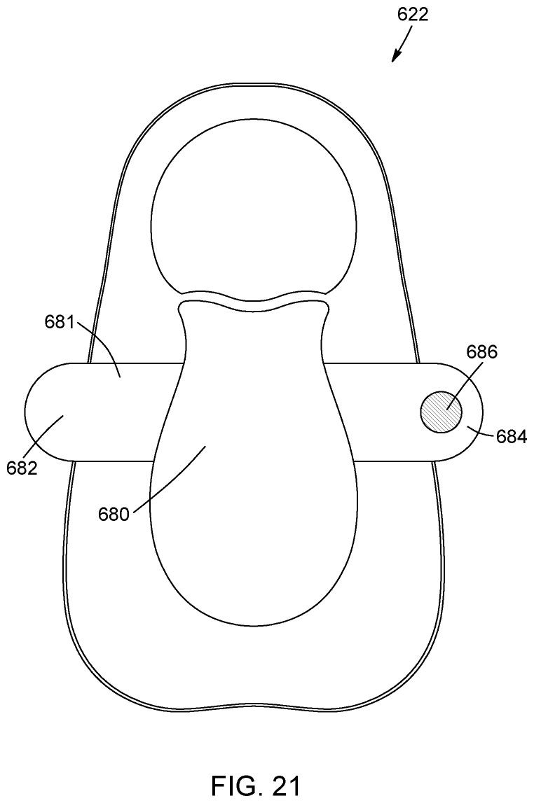

The wrapping cover 622, as represented in FIGS. 13 and 21, is dimensioned to fully wrap the infant once their head is supported on the plagiocephaly pillow assembly 702. In the embodiment shown, the wrapping cover 622 comprises two distinct elements (i.e. the body receiving section 680 configured to fully receive the body of the infant, and a wrapping piece 681 comprising opposed wrapping sections 682, 684), that might be secured (for instance sewn) to each other. The wrapping cover 622 also comprises a fastener 686 at one of the wrapping sections 684 (such as a hook and loop fastening system), to maintain the wrapping sections 682, 684 together when the infant is wrapped in the wrapping cover 622 and when the wrapping sections 682, 684 are folded on each other.

The plagiocephaly prevention set 700 also comprises a head-surrounding cushion 618. In the embodiment shown, the cushion 618 comprises support-fixing portions 619 on a bottom surface 621 of the cushion 618 for the cushion 618 to be better maintained in place when the plagiocephaly prevention set is in use.

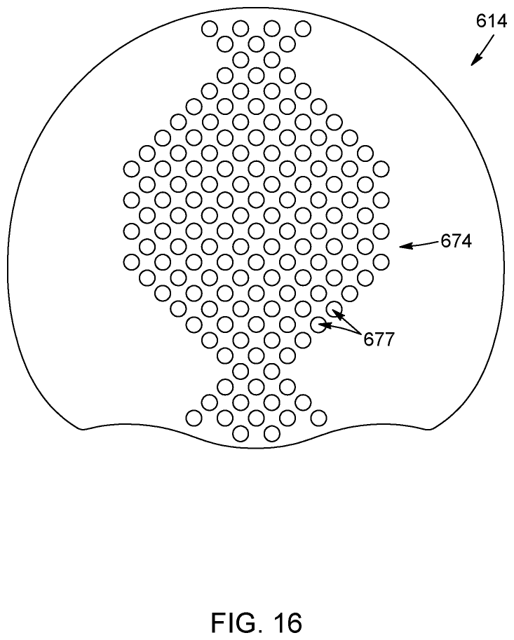

As represented in FIGS. 16 to 18 and 22A to 22C, similarly to the first embodiment of the plagiocephaly prevention set 100, base, first and second sets of depressions 654, 664, 674 are formed respectively in the base, first and second resilient layers 610, 612, 614. In the embodiment shown, the base, first and second sets of depressions comprise spaced-apart through holes 656, 667 and 677 extending substantially along an entirety of the thickness of the corresponding one of the base, first and second resilient layers 610, 612, 614.

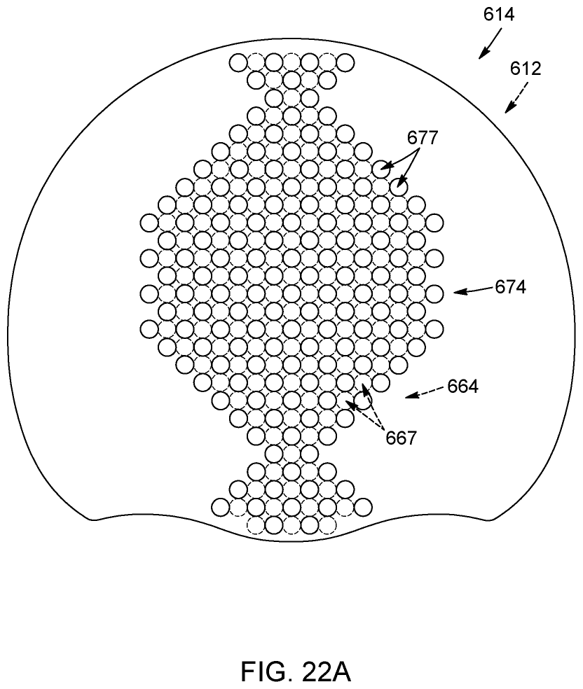

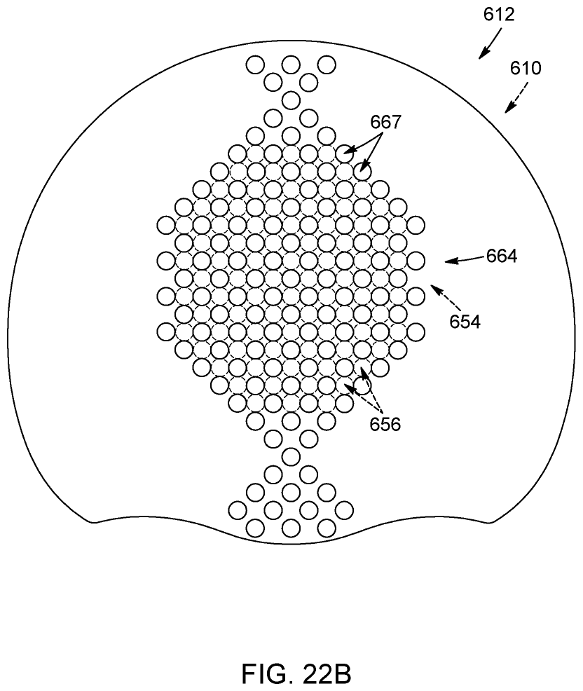

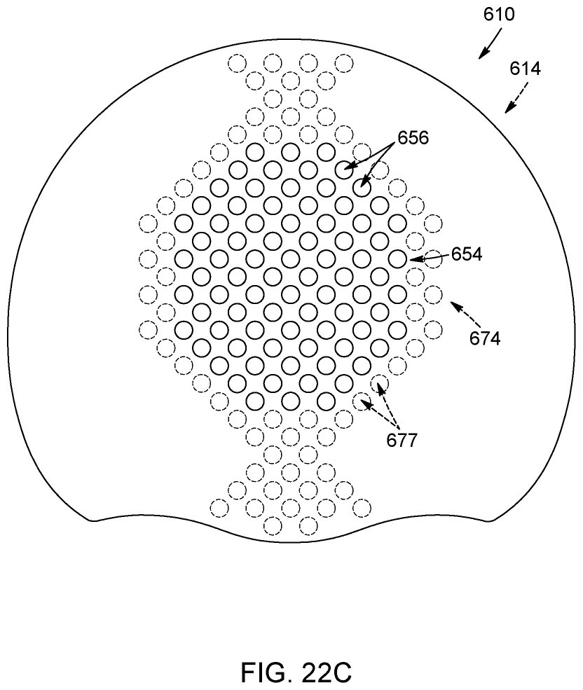

Similarly to the first embodiment, as represented in FIG. 22A in which the first set of depressions 664 is represented in dotted lines, the first and second sets of depressions 664, 674 comprising the through-holes 667, 677 are arranged so that, considered projected in a plane substantially parallel to at least one of the top and bottom surfaces of the first and second resilient layers 612, 614 (for instance in a plane substantially horizontal), at least some of the depressions of one of the first and second sets of depressions 664, 674 are not in register or aligned with any of the depressions of the other one of the first and second sets of depressions 664, 674 when the first and second resilient layers 612, 614 are superposed onto each other (for instance when at least some of the edges of the first and second resilient layers 612, 614 are substantially aligned with each other).

Moreover, as represented in FIGS. 22B and 22C in which the base set of depressions 654 comprising the through-holes 656 and the second set of depressions 674 comprising the through-holes 677 are respectively represented in dotted lines, at least some of the depressions formed in the base layer 610 are at least partially offset with regards to the depressions of the first set of depressions 664 and some of the depressions of the second set of depressions 674 are at least partially offset with regards to the depressions formed in the base layer 610.

### Method for Preventing Deformational Plagiocephaly

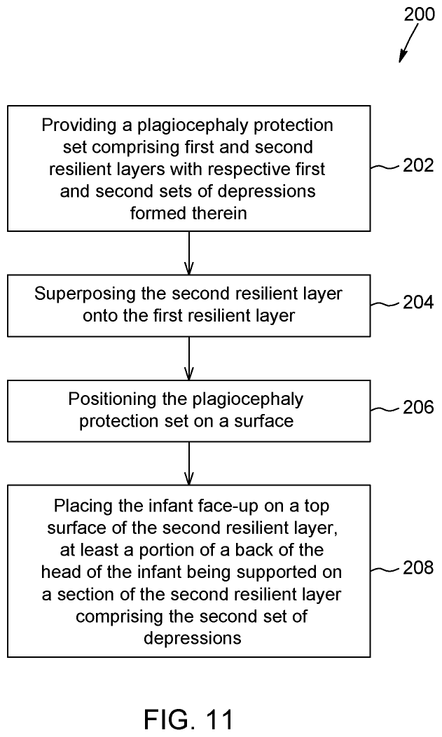

As represented in FIG. 11, the present disclosure also concerns a method 200 for preventing—or at least limiting—deformational plagiocephaly.

For instance, the method 200 is implemented using a plagiocephaly prevention set 700 according to the present disclosure.

In the embodiment shown, the method 200 firstly comprises a step 202 of providing a plagiocephaly prevention set 700 comprising a plagiocephaly pillow assembly 702 comprising a first resilient layer 612 with a first set of depressions 664 formed therein, a second resilient layer 614 comprising a top surface, a second set of depressions 674 being formed in the second resilient layer, at least some of the depressions of one of the first and second sets of depressions 664, 674 being at least partially offset with regards to the depressions of the other of the first and second sets of depressions when the first and second resilient layers are superposed onto each other.

The method 200 further comprises a step 204 of superposing the second resilient layer 614 onto the first resilient layer 612, steps 206 and 208, as represented in FIG. 23C, of respectively positioning the plagiocephaly prevention set 700 on a surface and of placing the infant I face-up on the top surface of the second resilient layer 614, at least a portion of a back of the head of the infant being supported on a section of the top surface of the second resilient layer comprising the second set of depressions.

In the embodiment shown, the step 202 further comprises providing a cushion 618 (or head-surrounding cushion 618). The method 200 further comprises a step of superposing the cushion 618 onto the top surface of the second resilient layer, as represented in FIG. 23A; and a step of at least partially surrounding the head of the infant with the cushion 618.

In the embodiment shown, the step 202 of the method 200 further comprises providing a protection cover 620 and arranging the protection cover 620 on the cushion and the plagiocephaly pillow assembly, as represented in FIG. 23B.

In the embodiment shown, the step 202 of the method 200 further comprises providing a wrapping cover and the method 200 further comprises a step of wrapping the wrapping cover 622 around a portion of the infant I, as represented in FIGS. 23D and 23E. In the embodiment shown, the wrapping cover 622 is arranged around a portion of a torso of the infant I, the arms of the infant I remaining outside the closed wrapping cover 622.

Several alternative embodiments and examples have been described and illustrated herein. The embodiments of the invention described above are intended to be exemplary only. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. It is understood that the invention may be embodied in other specific forms without departing from the central characteristics thereof. The present examples and embodiments, therefore, are to be considered in all respects as illustrative and not restrictive, and the invention is not to be limited to the details given herein. Accordingly, while the specific embodiments have been illustrated and described, numerous modifications come to mind. The scope of the invention is therefore intended to be limited solely by the scope of the appended claims.

1-84. (canceled)

85. A plagiocephaly pillow assembly for preventing deformational plagiocephaly of a head of an infant, the plagiocephaly pillow assembly comprising:
a first resilient layer with a first set of depressions formed therein;
a second resilient layer superposable onto the first resilient layer and comprising a top surface configured to support a portion of the head of the infant, a second set of depressions being formed in the second resilient layer;
wherein at least some of the depressions of one of the first and second sets of depressions are at least partially offset with regards to the depressions of the other of the first and second sets of depressions when the first and second resilient layers are superposed onto each other.

86. The plagiocephaly pillow assembly according to claim 85, wherein, considered in a projection plane substantially parallel to the top surface of the second resilient layer, at least some of the depressions of said one of the first and second sets of depressions are arranged at least partially between some of the depressions of the other of the first and second sets of depressions when the first and second resilient layers are superposed onto each other.

87. The plagiocephaly pillow assembly according to claim 85, wherein the first and second sets of depressions define respectively first and second depression patterns having each a width, a length and a surface area, the second depression pattern being different from the first depression pattern by at least one of the width, the length, the surface area of the first and second depression patterns and a number, dimensions and locations of the depressions.

88. The plagiocephaly pillow assembly according to claim 87, wherein at least one of the width, the length and the surface area of the second depression pattern is equal to or greater than the corresponding one of the width, the length and the surface area of the first depression pattern.

89. The plagiocephaly pillow assembly according to claim 85, wherein the first and second resilient layers have a thickness and wherein at least one of the first and second sets of depressions comprises spaced-apart through holes extending substantially along an entirety of the thickness of the corresponding one of the first and second resilient layers.

90. The plagiocephaly pillow assembly according to claim 87, comprising a base resilient layer, the first resilient layer being superposable onto the base resilient layer, wherein a base set of depressions is formed in the base resilient layer and defines a base depression pattern and wherein the base resilient layer has a thickness, the base set of depressions comprising spaced-apart through holes extending along an entirety of the thickness of the base resilient layer.

91. The plagiocephaly pillow assembly according to claim 90, wherein the base depression pattern has a width, a length and a surface area, wherein at least one of the width, the length and the surface area of the first depression pattern is equal to or greater than the corresponding one of the width, the length and the surface area of the base depression pattern.

92. The plagiocephaly pillow assembly according to claim 85, wherein the plagiocephaly pillow assembly defines a height and has a density decreasing upwardly along the height of the plagiocephaly pillow assembly.

93. The plagiocephaly pillow assembly according to claim 92, wherein the plagiocephaly pillow assembly has an Indentation Load Deflection (ILD) decreasing upwardly along the height of the plagiocephaly pillow assembly.

94. The plagiocephaly pillow assembly according to claim 90 wherein said at least one of the base, first and second resilient layers is at least partially made of a polyurethane foam, an open-cell foam or a visco-elastic foam.

95. The plagiocephaly pillow assembly according to claim 85, comprising a bottom edge, wherein at least first and second shoulder-receiving recesses are formed in the bottom edge.

96. The plagiocephaly pillow assembly according to claim 85, wherein the first and second resilient layers are removably superposable onto each other.

97. The plagiocephaly pillow assembly according to claim 85, comprising a pillowcase shaped and dimensioned to receive at least the first and second resilient layers when the first and second resilient layers are superposed onto each other and wherein the pillowcase comprises a bottom surface and at least one support-fixing portion secured to the bottom face.

98. A plagiocephaly pillow assembly for preventing deformational plagiocephaly of a head of an infant, the plagiocephaly pillow assembly comprising:
a first resilient layer with a first set of depressions formed therein;
a second resilient layer superposable onto the first resilient layer and comprising a top surface configured to support a portion of the head of the infant, a second set of depressions being formed in the second resilient layer;
wherein a density of the first resilient layer is different from a density of the second resilient layer.

99. The plagiocephaly pillow assembly according to claim 98, wherein, considered in a projection plane substantially parallel to the top surface of the second resilient layer, at least some of the depressions of one of the first and second sets of depressions are arranged at least partially between some of the depressions of the other of the first and second sets of depressions when the first and second resilient layers are superposed onto each other.

100. The plagiocephaly pillow assembly according to claim 98, wherein the first and second sets of depressions define respectively first and second depression patterns having each a width, a length and a surface area, wherein at least one of the width, the length and the surface area of the second depression pattern is equal to or greater than the corresponding one of the width, the length and the surface area of the first depression pattern.

101. The plagiocephaly pillow assembly according to claim 98, comprising a base resilient layer, the first resilient layer being superposable onto the base resilient layer, wherein a density of the base resilient layer is bigger than the density of at least one of the first and second resilient layers.

102. The plagiocephaly pillow assembly according to claim 101, wherein the plagiocephaly pillow assembly defines a height and has an Indentation Load Deflection (ILD) decreasing upwardly along the height of the plagiocephaly pillow assembly.

103. A plagiocephaly prevention set comprising:
a plagiocephaly pillow assembly according to claim 85; and
at least one of a cushion superposable onto the top surface of the second resilient layer and a wrapping cover shaped and dimensioned to at least partially wrap the infant once the head of the infant is supported on the top surface of the second resilient layer.

104. A method for preventing deformational plagiocephaly of a head of an infant, comprising:
providing a plagiocephaly prevention set comprising:
a first resilient layer with a first set of depressions formed therein;
a second resilient layer comprising a top surface, a second set of depressions being formed in the second resilient layer;
at least some of the depressions of one of the first and second sets of depressions being at least partially offset with regards to the depressions of the other of the first and second sets of depressions when the first and second resilient layers are superposed onto each other;
superposing the second resilient layer onto the first resilient layer;
positioning the plagiocephaly pillow assembly on a surface; and
placing the infant face-up on the top surface of the second resilient layer, at least a portion of a back of the head of the infant being supported on a section of the top surface of the second resilient layer comprising said second set of depressions.
